(19) United States
(12) Patent Application Publication
(10) Pub. No.: US 2014/0357526 A1
(43) Pub. Date: Dec. 4, 2014

(54) HYDROGEL-MEDIATED TISSUE ANALYSIS

(71) Applicant: Vanderbilt University, Nashville, TN (US)

(72) Inventors: Richard Caprioli, Brentwood, TN (US); Glenn A. Harris, Nashville, TX (US); Joshua J. Nicklay, Nashville, TN (US)

(73) Assignee: Vanderbilt University, Nashville, TN (US)

(21) Appl. No.: 14/293,397

(22) Filed: Jun. 2, 2014

Related U.S. Application Data
(60) Provisional application No. 61/829,524, filed on May 31, 2013.

(51) Int. Cl. G01N 33/68; C12Q 1/37

(52) U.S. Cl. CPC G01N 33/6848; C12Q 1/37; G01N 33/6851; G01N 33/6887; G01N 2570/00; G01N 2560/00
USPC 506/12; 435/23; 435/15; 435/21; 435/19

(57) ABSTRACT

A method for analyzing the polypeptide content of animal tissue is described. The method includes the steps of (a) providing an animal tissue specimen; (b) depositing one or more portions of a hydrogel mixture including a protease on spatially discrete portions of the animal tissue specimen; (c) allowing sufficient time to pass for animal tissue under the hydrogel mixture to be form a digested mixture of animal tissue and hydrogel mixture; (d) removing the digested mixture from the animal tissue and extracting the polypeptides from the digested mixture to provide an extract; and (e) analyzing the polypeptide content of the extract by mass spectrometry.

This application claims the benefit of U.S. Provisional Application Ser. No. 61/829,524, filed May 31, 2013, the disclosure of which is incorporated by reference herein.

### STATEMENT REGARDING FEDERALLY SPONSORED RESEARCH

This invention was made with government support under grant 5P41RR031461-02 awarded by the National Center for Research Resources and under grant 8 P41 GM103391-02 awarded by the National Institute of General Medical Sciences from the National Institutes of Health. The Government has certain rights in this invention.

### BACKGROUND

Tissue analyses, including histomorphological and immunohistochemical approaches, form the basis for most diagnostic analyses in anatomic pathology. Highly standardized approaches and rigorous training regimens have been instituted to ensure that these morphological approaches to disease characterization deliver a high standard of care; therefore, many patient lives are saved annually using these approaches. However, in spite of these safeguards, there still exist situations for which the current methods do not provide a definitive diagnosis. For these unfortunate cases, new technological approaches that incorporate molecular analysis would add significant value to the diagnostic process. The development of proteomics and mass spectrometry technologies during the previous decade has enabled rapid and specific protein analyses. These technical advances now provide the opportunity to contribute molecular information with high chemical and spatial specificity at sufficient throughput to aid in the histopathological evaluation of patient specimens.

Protein analysis and identification are traditionally performed through the use of one of two different strategies. Using gel electrophoresis, proteins are separated in one or two dimensions (1D/2D) on a gel and enzymatic digestion is performed in-gel, a time-consuming and manual process. In a second solution-based approach, proteins or peptides are separated chromatographically using on-line LC systems and the proteins are digested in solution prior to the chromatographic analysis. Capelo et al., Anal. Chim. Acta 650, 151-9 (2009). The in-solution approach tends to be the simplest in terms of sample handling and speed, but the digestion step is still the most time-consuming step in the sample preparation workflow. Another disadvantage to this approach is the requirement for sample homogenization. Common proteomics workflows such as those described require microgram to milligram quantities of proteins be extracted from the tissue to provide sufficient material to perform the analysis. This requires the homogenization of the bulk sample, a step that can significantly diminish the possibility of studying specific cells in relation to their native environment in the tissue.

Histology-guided approaches to the sampling or analysis of tissues have been developed that can overcome these problems. For example, groups have reported the use of laser microdissection (LM) to sample specific cell types from tissues (both fresh and formalin fixed) (Mukherjee et al., Methods in Mol. Biol. 1002, 71-83 (2013)) and subsequent analysis of these samples using a variety of genomics and proteomics approaches. This approach has been utilized to study the molecular content in histologically distinct tissue regions in a variety of disease states. Jain et al., Am. J. of Kidney Disease 63, 324-328 (2014); Xing et al., Oncology Reports 31, 634-640 (2014). Furthermore, there now exists a proteomics-based diagnostic test that combine LM with LC-MS/MS to type specific amyloid proteins in patient biopsies. Vrana et al., Blood 114, 4957-9 (2009). In spite of the advantages and the utility of LM as a sampling approach for proteomics of tissue specimens, throughput is very limited, making it difficult to anticipate analyzing more than a few samples at one time.

One alternative is to perform the digestion directly on cryosectioned tissue, then identify the proteins or their constituent peptides directly from the tissue surface via tandem MS (MS/MS) and accurate mass measurements. The bottom-up approach, including in situ proteolytic digestion, is often used to identify a pool of proteins from which all potential biomarkers are most likely derived. Groseclose et al., J Mass Spectrom., 42(2), 254-62 (2007). However, on-tissue protein identification can be laborious. Many traditional proteomic methodologies to identify proteins may involve one of several approaches such as microextraction with solvents from the tissue surface, tissue homogenization using multiple tissue specimens or laser capture microdissection (LCM) of the regions of interest on a single tissue specimen. Franck et al., Mol. Cell. Proteomics 8, 2023-33 (2009); Xu et al., J. Am. Soc. Mass Spectrom. 13, 1292-7 (2002). All of these approaches require an overnight digestion, an approach that can be problematic for analyses of such small volumes on-tissue surfaces where evaporation and delocalization of the solvent can stop the digestion prematurely.

The enzymatic digestion step is commonly the bottleneck of the workflows used in proteomics. Previously, many research teams have developed new protocols for protein digestion and identification that are designed to reduce the sample handling while increasing sample throughput. Santos et al., Proteomics 13, 1423-7 (2013). These two goals have been achieved by reducing the total time of the entire workflow or increasing the number of samples treated at the same time. Many tools have been successfully used to accelerate the enzymatic digestion of proteins: for example, heating, microspin columns, ultrasonic energy, high pressure, infrared energy, alternating electric field or microwave. Juan et al., Proteomics 5, 840-2 (2005). While microwave assisted proteolytic digestion has traditionally been implemented in solution, there is a growing trend to use heterogeneous systems for on-tissue digestion in which enzyme is carried within hydrogels or adsorbed on solid supports. Fiddes et al., Biomicrofluidics 6, 14112-1411211 (2012); Luk et al., Proteomics 12, 1310-8 (2012). Molecular hydrogels have attracted extensive research interest recently because of their great potential for tissue engineering, migration of organic and inorganic material, drug delivery as well as a miniaturized method for application on biological samples. Toledano et al., J. Am. Chem. Soc. 128, 1070-1 (2006); Li et al., Chem. Commun. (Carob). 48, 6175-7 (2012).

Improvements and complementary methods are needed to address difficulties and challenges of the on-tissue identification process. More user-friendly approaches should be adopted to obviate the need of costly robotic liquid extraction, matrix deposition and tissue isolating instruments.

### SUMMARY

Proteomics is an extremely powerful tool in examining cellular function, and provides a complementary analysis to genomics efforts. While it is somewhat more complicated to examine protein expression profiles, mass spectrometry (MS), because of its extreme selectivity and sensitivity, has now become a favored tool in the global examination of protein expression. However, a limitation on any analysis of this nature is the need to interrogate molecular changes in discrete tissue samples while permitting high throughput.

Traditional methods of examining proteomes with MS involve homogenizing small samples of tissues, using separative techniques such as 2D gels or liquid chromatography, which are followed by MS for detection. Although this method gives adequate results, it is tedious, labor intensive and destroys any spatial fidelity in the sample due to the homogenization process. Therefore, current approaches to MS quantification of protein expression require substantial improvements in sample processing and utilization.

The present inventors have developed a method for analyzing protein expression in situ, i.e., directly in intact tissues and within discrete areas thereof. In particular embodiments, one or more portions of a hydrogel mixture including a protease are deposited on spatially discrete portions of an animal tissue specimen. The protease of the hydrogel mixture results in the formation of a digested mixture of animal tissue and hydrogel mixture. The digested mixture is then removed from the animal tissue and polypeptides are extracted to provide an extract. The polypeptide content of the extract is then analyzed by mass spectrometry.

### BRIEF DESCRIPTION OF THE FIGURES

FIG. 1 provides a scheme showing hydrogel-mediated proteomic digestion and extraction workflow beginning with a) hydrogel synthesis on a laser printed piece of chromatography paper, b) on-tissue placement of gel for proteolysis and incubation, c) solvent extraction in aqueous and organic solvents, and analysis of reconstituted extracts with d) MALDI MS and/or e) LC-MS/MS.

FIG. 2 provides mass spec. images showing MALDI MS profile analysis of 2% (by volume) of the reconstituted hydrogel mediated digestion extracts from rat brain cerebrum (12 μm thickness). Linear mode full scan spectra (m/z 1500-10000, 2000 shots summed) from a) trypsin loaded hydrogel placed off-tissue (blank), b) on-tissue hydrogel without trypsin (control) and c) an on-tissue trypsin hydrogel (digest).

FIG. 3 provides images showing a) Image overlay of three PC lipid species analyzed using ion mobility MALDI IMS:PC 36:1, PC 34:1, and PC 36:4. Scale bars are 2 mm. On-tissue MS/MS spectra for the precursor b) protonated ion of PC 36:1 at m/z 788.62, c) potassiated ion of PC 34:1 at m/z 798.54 and d) sodiated ion PC 36:4 at m/z 804.55 were obtained prior to on-tissue hydrogel digestion.

FIG. 4 provides images showing a) hematoxylin and eosin (H & E) stain of an untreated rat cerebellum region with outlined (circle) region of where the hydrogel was placed on the serial digested tissue (scale bar 250 μm) and b) the H & E stain of the serial sectioned, previously imaged (FIG. 3) and hydrogel digested rat cerebellum (scale bar 50 μm).

FIG. 5 provides a scheme showing the workflow of hydrogel discs fabrication and of the on-tissue digestion is here presented. a) a bis-polyacrylamide solution is prepared and allowed to polymerize for 20 min. Once the gel is formed in a glass petri dish (5 cm diameter), hydrogel discs are created using a punch biopsy tool at 1 mm diameter. About 150 hydrogel discs are created per 5 cm dish. The discs are then placed into separate eppendorf tubes, fully dried and stored at 4° C. until use. To start the digestion experiment, a hydrogel disc is swelled in few microliters of the enzyme solution and then placed on the region of interest within the tissue specimen. b) the tissue specimen with the hydrogel disc on the surface is heated in the microwave oven for 2 min to allow the digestion. After that, the disc is removed from the tissue surface and placed into an eppendorf tube. A solvent extraction is carried out and, once dried, the solution is reconstituted as described herein and ready to be used for both MALDI MS profiling as well as for LC-MS/MS analysis followed by data base search for protein identification.

FIG. 6 provides a scheme showing a workflow of the histology-directed on-tissue enzymatic digestion; a) H&E of a fresh frozen rat brain tissue specimen (cryosectioned at 8 μm), stained for histological evaluation and localization of the brain thalamic region. Enzymatic digestions were performed depositing the hydrogel disc embedded with trypsin on the thalamic region and then incubating the tissue specimen into the microwave for 2 min; further, a consecutive cut tissue specimen was incubated in an oven; b) the rat brain punch biopsy was obtained from the thalamic region at the same diameter of the hydrogel disc (1 mm) and then cryosectioned at 8 μm. Protein digestion experiments were carried out using the hydrogel device as well as manual spotting the enzyme solution and homogenizing tissue specimens within a conventional oven.

FIG. 7 provides a MALDI MS spectra of the solvent extracted digested peptides obtained from the first two hydrogel experiments, carried out via microwave (grey) and oven (black). The resulting profiles display a high degree similarity in the ions present in the mass range 500-4000 Da.

FIG. 8 provides a MALDI MS spectra of the three technical replicates of microwave assisted hydrogel mediated on-tissue digested extracts. The resulting profiles display a high degree similarity in both the ions present and their relative abundance in the mass range 500-4000 Da.

FIG. 9 provides a Venn diagrams summarizing the number of identified proteins for N=3 on-tissue digestion experiments (5% FDR, ≧2 unique peptides and p value <0.05). a) shows the number of identified proteins within the microwave digested extracts and the oven incubation digested extracts, both carried out using the hydrogel disc placed onto the rat brain thalamic region; b) displays the number of identified proteins within the experiments performed using the 1 mm diameter tissue specimen from the same region of interest within the rat brain biopsy, using the hydrogel disc and homogenizing the tissue specimens respectively.

FIG. 10 provides bar graphs shows the number of protein groups and of distinct peptides obtained from the hydrogel experiments carried out via microwave and oven (a) and from the experiments performed on the 1 mm diameter rat brain tissue specimens from thalamic region (h). The results are expressed as mean±SD (N=3). Data are averaged from N=3 replicated experiments per class. Asterisk denotes comparisons found to be statistically significant, p<0.05.

### DETAILED DESCRIPTION

A method for analyzing the polypeptide content of animal tissue is described. The method includes the steps of (a) providing an animal tissue specimen; (b) depositing one or more portions of a hydrogel mixture including a protease on spatially discrete portions of the animal tissue specimen; (c) allowing sufficient time to pass for animal tissue under the hydrogel mixture to be form a digested mixture of animal tissue and hydrogel mixture; (d) removing the digested mixture from the animal tissue and extracting the polypeptides from the digested mixture to provide an extract; and (e) analyzing the polypeptide content of the extract by mass spectrometry.

### DEFINITIONS

Unless otherwise defined, all technical and scientific terms used herein have the same meaning as commonly understood by one of ordinary skill in the art to which this invention pertains. In case of conflict, the present specification, including definitions, will control.

The terminology as set forth herein is for description of the embodiments only and should not be construed as limiting the application as a whole. Unless otherwise specified, “a,” “an,” “the,” and “at least one” are used interchangeably. Furthermore, as used in the description of the application and the appended claims, the singular forms “a”, “an”, and “the” are inclusive of their plural forms, unless contraindicated by the context surrounding such. Furthermore, the recitation of numerical ranges by endpoints includes all of the numbers subsumed within that range (e.g., 1 to 5 includes 1, 1.5, 2, 2.75, 3, 3.80, 4, 5, etc.).

As used herein, the term “polypeptide” refers to an oligopeptide, peptide, or protein, or to a fragment, portion, or subunit of any of these, and to naturally occurring or synthetic molecules. The term “polypeptide” also includes amino acids joined to each other by peptide bonds or modified peptide bonds, i.e., peptide isosteres, and may contain any type of modified amino acids. The term “polypeptide” also includes peptides and polypeptide fragments, motifs and the like, glycosylated polypeptides, all “mimetic” and “peptidomimetic” polypeptide forms, and retro-inversion peptides (also referred to as all-D-retro or retro-enantio peptides). Generally, a peptide has less than 30 amino acids, whereas a protein has more than 30 amino acids, though this is an approximate dividing line between the two.

A subject can be any animal, and animal tissue described herein can be obtained from any type of subject. In some embodiments, the subject is a mammal, such as a domesticated farm animal (e.g., cow, horse, pig) or pet (e.g., dog, cat). More preferably, the subject is a human. When the subject is a human, the subject may also be referred to as a patient, particularly when the subject is being evaluated in a medical environment or by medical personnel.

In one aspect, the present invention provides a method for analyzing the polypeptide content of animal tissue. The method includes the steps of (a) providing an animal tissue specimen; (b) depositing one or more portions of a hydrogel mixture including a protease on spatially discrete portions of the animal tissue specimen; (c) allowing sufficient time to pass for animal tissue under the hydrogel mixture to be form a digested mixture of animal tissue and hydrogel mixture; (d) removing the digested mixture from the animal tissue and extracting the polypeptides from the digested mixture to provide an extract; and (e) analyzing the polypeptide content of the extract by mass spectrometry.

The method includes the step of depositing one or more portions of a hydrogel mixture including a protease onto spatially discrete portions of the animal tissue specimen. A hydro gel may be defined as a three-dimensional, hydrophilic or amphiphilic polymeric network capable of taking up large quantities of water. The networks are composed of homopolymers or copolymers, are insoluble due to the presence of covalent chemical or physical (ionic, hydrophobic interactions, entanglements) crosslinks. The crosslinks provide the network structure and physical integrity. Hydrogels exhibit a thermodynamic compatibility with water that allows them to swell in aqueous media.

In some embodiments, the hydrogel is prepared by crosslinking hydrophilic biopolymers or synthetic polymers. Examples of the hydrogels formed from physical or chemical crosslinking of hydrophilic biopolymers, include but are not limited to, hyaluronans, chitosans, alginates, collagen, dextran, pectin, carrageenan, polylysine, gelatin or agarose. Examples of hydrogels based on crosslinked synthetic polymers include but are not limited to (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL(G)A-PEO-PL(G)A copolymers, poly(ethylene imine), etc. See A. S Hoffman, Adv. Drug Del. Rev 43, 3-12 (2002). In some embodiments, the hydrogel is an alginate or polyacrylamide hydrogel.

In some embodiments, the hydrogel is an ionotropic hydrogel. When a polyelectrolyte is combined with a multivalent ion of the opposite charge, it may form a physical hydrogel known as an ‘ionotropic’ hydrogel. Calcium alginate is an example of an ionotropic hydrogel. Further, when polyelectrolyte of opposite charges is mixed, they may gel or precipitate depending on their concentrations, the ionic strength, and pH of the solution. The products of such ion crosslinked systems are known as complex coacervates, polyion complexes, or polyelectrolyte complexes.

In some embodiments, one or more portions of the hydrogel including the protease are placed directly on the animal tissue specimen. The portions of hydrogel can vary in size and shape. In some embodiments, the hydrogel portions are drops or discs. In some embodiments, the drops or discs have a diameter ranging from about 100 μm to about 5 mm, while in other embodiments, the drops or discs have a diameter ranging from about 250 μm to about 1 mm, or from about 400 to 700 μm. The size and shape of the hydrogel portion determines the size and shape of the animal tissue section within the specimen that is evaluated using that portion.

In some embodiments, the hydrogel is first applied in a low viscosity state to a paper template and thereafter converted into a high viscosity gel when applied. Use of a paper template facilitates preparing hydrogel portions in whatever size and shape is desired. For example, methods of forming hydrogels using paper are described in International Publication No. WO 2009/121038, entitled “Shaped Films of Hydrogels Fabricated Using Templates of Patterned Paper,” filed Mar. 27, 2009, incorporated in its entirety by reference. The paper can be printed with the desired shapes corresponding to that of the portion of hydrogel that is applied to the animal tissue specimen. By using hydrophobic ink, hydrophobic boundaries can be created on the paper surface. A preferred type of paper is chromatography paper. In one exemplary method, an ionotropic hydrogel is formed by contacting the substrate with a solution of one or more gelling agents, including but not limited to metallic ions, such as Pb²⁺, Ba²⁺, Fe³⁺, Al³⁺, Cu²⁺, Cd²⁺, Ho³⁺, Ca²⁺, Zn²⁺, Co²⁺, Ni²⁺, Mn²⁺, and Mg²⁺, and contacting the substrate with a hydrogel precursor such as alginic acid (AA), carboxymethylcellulose (CMC), -carrageenan, poly(galacturonic acid) (PG), or acrylamide/bisacrylamide. The interaction of the gelling agent, e.g., ion, with the hydrogel precursor results in gelation of the hydrogel.

Typically, the method of tissue analysis involves obtaining tissue samples from a variety of locations on the subject or a particular animal tissue specimen. In such embodiments, a plurality of portions of hydrogel mixture are deposited to provide a plurality of extracts for analysis by mass spectrometry. For example, 5 or more, or 10 or more, or 50 or more portions of hydrogel mixture can be deposited. The portions of hydrogel mixture should be deposited on spatially discrete regions of the animal tissue specimen. A spatially discrete region of animal tissue is a region that does not overlap with another region that is being analyzed. In some embodiments, the portions of hydrogel mixture can be deposited in a regular pattern in order to facilitate conducting and/or automating the assay. For example, the portions of hydrogel can be positioned in regularly spaced rows and columns. When conducting the method of tissue analysis, it can be useful to compare the polypeptide content from a plurality of spatially discrete regions of the animal tissue.

### Animal Tissue Specimens

In accordance with the present invention, intact tissue samples are obtained by standard methodologies for use as animal tissue specimens. The tissue samples must be of a sufficient size to permit creation of a plurality of microregions, e.g., at least 1 micron to several millimeters, including sizes in between, such as 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 m, 50 m, 60 μm, 70 μm, 80 μm, 90 μm, 100 μm, 125 μm, 150 μm, 175 μm, 200 μm, 250 μm, 275 μm, 300 μm, 400 μm, 450 tun, 500 μm, 600 μm, 700 μm, 800 μm, 900 μm, 1000 μm, 2 mm, 3 mm, 4 mm and 5 mm. In some embodiments, the tissue specimen has a thickness ranging from about 5 μm to about 50 μm. In some embodiments, the animal tissue specimen is cryosectioned animal tissue, which can be prepared using a cryostat.

Any type of animal tissue can be analyzed for its polypeptide content. The animal tissue evaluated can be healthy tissue, or it can be tissue that is diseased or injured. Examples of animal tissue suitable for evaluation using the present invention include heart tissue, liver tissue, kidney tissue, prostate tissue, breast tissue, ovary tissue, uterine tissue, skin tissue, lung tissue, brain tissue, colon tissue, pancreatic tissue, and muscle tissue.

Biopsy procedures for obtaining the specimen will generally involve the sterility required of surgical operations, even though the tissues being sample are from cadavers or animals that will be sacrificed. For internal tissues, incisions will be made proximal to the tissue of interest, followed by retraction, excision of tissue and surgical closing of the incision. Superficial tissue sites are accessed by simple excision of the available tissue.

Tissue specimens should be handled such that (a) the integrity of the tissue is maintained and (b) that the cells within the tissue, particularly those in the region(s) where hydrogel portions will be placed are not damaged. Appropriate physiologic buffers are generally applied to the tissue, or the tissues are immersed therein. The tissue may also be cooled to appropriate temperatures for limited periods of time. Steps should be taken to ensure that apoptosis or other cellular degradation will not be induced in the tissue specimen.

In some embodiments, pretreatment with of tissues prior to application of the hydrogel may prove advantageous. On particularly useful pretreatment is an ethanol wash, optionally followed by a storage period of minutes to hours. In tests on several tissue types, improved well formation was observed using this approach. Also, the delivery of matrix in a solvent comprising 10% acetonitrile, 60% water, 30% isopropanol and 0.5% acetic acid provided improved results.

### Protein Digestion

A feature of the present invention is that tissue digestion to release polypeptides (i.e., proteins and/or peptides) from the tissue is carried out directly on the animal tissue surface (i.e., “in situ”) using an enzyme included in the hydrogel. Preferably, the enzyme is a protease (i.e., proteolytic enzyme or proteinase). Proteases are involved in digesting long protein chains into shorter fragments by splitting the peptide bonds that link amino acid residues. Examples of proteases include serine proteases, threonine proteases, cysteine proteases, aspartate proteases, glutamic acid proteases, and metalloproteases. Protease digestion serves to release polypeptides from the animal tissue surface, and to digest proteins to facilitate analysis my mass spectrometry.

Most commonly digestions are carried out with the proteases trypsin or lysine specific proteinases, because these enzymes are reliable, specific and produce a suitable number of peptides. The next most common digestion is at aspartate or glutamate using endoproteinase Glu-C or endoproteinase Asp-N. Chymotrypsin is sometimes used, although it does not have a well defined specificity. Proteinases of broad specificity may generate many peptides, and the peptides may be very short.

In some embodiments, the hydrogel mixture is heated during formation of the digested mixture. Heating increases the rate at which digestion of the tissue occurs, thereby decreasing the amount of time necessary for a sufficient time to pass for the hydrogel mixture to form a digested mixture. The hydrogel mixture should be heated to a temperature above room temperature. For example, in some embodiments, it is preferable to heat the hydrogel mixture to a temperature from about 35° C. to about 60° C. for a time from about 2 to about 12 hours. The heating should be sufficient to accelerate digestion, without destroying the protease, polypeptide, or animal tissue. A variety of means can be used to heat the digested mixture. For example, the digested mixture can be heated by placing the tissue bearing the hydrogel mixture into an oven. Preferably the animal tissue is held within an air-tight container during heating in order to prevent drying of the hydrogel during heating.

In some embodiments, the hydrogel mixture is heated using microwave radiation. Use of microwave radiation has the advantage of significantly increasing the speed of protein digestion, which has the effect of further decreasing the amount of time to provide a sufficient time to pass for the hydrogel mixture to form a digested mixture. The microwave radiation can be any suitable wavelength (e.g., from about 0.5 to 5 GHz) and any suitable wattage (e.g., from about 300 to 3,000 watts). Because of the efficiency of microwave heating, a sufficient time for digestion can be from 1 minute to 1 hour, with typical settings providing digestion in from about 1 minute to about 5 minutes.

### Extraction and Sample Preparation

Once one has obtained a digested mixture of animal tissue and hydrogel, an extraction step is carried out to extract the polypeptides from the digested mixture in order to provide an extract for analysis by mass spectrometry. The extraction step can include an organic extraction and/or an aqueous extraction. Extraction will shrink and swell the hydrogel in order to release polypeptides within the digested mixture, and can also separate the polypeptides, which migrate to the aqueous phase, from other components in the extraction mixture.

As the collected samples are to be prepared for proteomic analysis, proteins should be extracted from lipids, metabolites, and other non-proteinaceous compounds, which may interfere with downstream procedures. Various chemical precipitation methods are available for protein isolation; these include acetone, trichloroacetic acid (TCA), ethanol, isopropanol, chloroform/methanol, and ammonium sulfate. The efficiency of protein precipitation varies among different organic solvents. For example, acetone has been determined to precipitate more acidic and hydrophilic proteins, whereas ultracentrifugation fractionates more basic, hydrophobic, and membrane proteins. Thongboonkerd et al., Kidney Int. 62(4):1461-9 (2002). Alternatively, chloroform methanol extraction has been used to successfully extract hydrophobic proteins. Stark et al., Eur J. Biochem. 266(1):209-14 (1999). Precipitation strategies can be optimized for a particular sample type.

In some embodiments, it may be preferable to treat the polypeptide extract with various enzymes (lipases, collagenases, proteases, nucleases) to further purify the sample. Examples of these treatments are provided below.

### Lipases

In some embodiments, lipases may be used to further degrade lipid contaminants. Most lipases exhibit the catalytic triad Ser-Asp-His (an exception being geotrichium candidum which has Ser-Glu-His). Lipases have been isolated from a wide variety of mammalian and microbial sources. The mammalian lipases can be split into four groups, the hepatic lingual, gastric and pancreatic lipase and microbial lipases into bacterial and fungal. Very little homology has been found within the known sequences, the most conserved feature being the consensus sequence G×S×G found in the substrate binding site. The above-mentioned catalytic triad (Ser-Asp-His) is also a highly conserved. However, this is common to all esterases, not just lipases, as is the α/β hydrolase fold.

Known lipases include triacylglycerol lipase (triglyceride lipase; tributyrase) phospholipase A2 (phosphatidylcholine 2-acylhydrolase, lecithinase A, phosphatidase, phosphatidolipase) lysophospholipase (lecithinase B, lysolecithinase, phospholipase B) acylglycerol lipase (monoacylglycerol lipase) galactolipase, phospholipase Al, lipoprotein lipase (clearing factor lipase, diglyceride lipase, diacylglycerol lipase) dihydrocoumarin lipase, 2-acetyl-1-alkylglycerophosphocholine esterase (1-alkyl-2-acetylglycerophosphocholine esterase, platelet-activating factor acetylhydrolase, PAP acetylhydrolase, PAF 2-acylhydrolase, LDL-associated phospholipase A2 LDL-PLA(2)), phosphatidylinositol deacylase (phosphatidylinositol phospholipase A2) phospholipase C (lipophosphodiesterase I, Lecithinase C, Clostridium welchii α-toxin, Clostridium oedematiens β- and γ-toxins) phospholipase D, (lipophosphodiesterase II, lecithinase D, choline phosphatase), phosphoinositide phospholipase C (triphosphoinositide phosphodiesterase, phosphoinositidase C, 1-phosphatidylinositol-4,5-bisphosphate phosphodiesterase, monophosphatidylinositol phosphodiesterase. phosphatidylinositol phospholipase C, PI-PLC, 1-phosphatidyl-D-myo-inositol-4,5-bisphosphate inositoltrisphosphohydrolase), alkylglycerophosphoethanolamine phosphodiesterase. (lysophospholipase D), glycosylphosphatidylinositol phospholipase D (GPI-PLD, glycoprotein phospholipase D, phosphatidylinositol phospholipase D, phosphatidylinositol-specific phospholipase D, phosphatidylinositol-glycan-specific phospholipase D), phosphatidylinositol diacylglycerol-lyase (1-phosphatidylinositol phosphodiesterase, monophosphatidylinositol phosphodiesterase, phosphatidylinositol phospholipase C, 1-phosphatidyl-D-myo-inositol inositolphosphohydrolase (cyclic-phosphate-forming)), glycosylphosphatidylinositol diacylglycerol-lyase ((glycosyl)phosphatidylinositol-specific phospholipase C, GPI-PLC, GPI-specific phospholipase C, VSG-lipase, glycosyl inositol phospholipid anchor-hydrolyzing enzyme, glycosylphosphatidylinositol-phospholipase C, glycosylphosphatidylinositol-specific phospholipase C, variant-surface-glycoprotein phospholipase C).

### Collagenases

Collagen is the most abundant protein in vertebrates, and occurs in almost every tissue. However, for many applications, is it necessary to remove collagen in order to analyze other proteins in a sample. Moreover, analysis of collagen may be of only limited interest. As a result, methods for the removal of collagen are regularly employed. Collagenases are enzymes that are able to cleave the peptide bonds in triple helical collagen molecules. Collagenases from Clostridium histolyticum have been known and studied for decades. Clostridopeptidase and clostripain activities also are associated with some collagenase preparations.

### Nucleic Acid Removal

Elimination of nucleic acids from sample prior to analysis can be achieved by chemical or enzymatic means. Chemical removal involves precipitation methods that employ polyethyleneimine (PEI) or streptomycin sulfate precipitation, followed by centrifugation.

Alternatively, enzymes that specifically degrade DNA and/or RNA may be used to remove these molecules. Benzonase is a genetically engineered endonuclease from Serratia marcescens. The protein is a dimer of two 30 kDa subunits. The enzyme degrades all forms of DNA and RNA, including single-stranded, double-stranded, linear and circular molecules, and is effective over a wide range of operating conditions. Some sequence specificity has been identified, with GC-rich regions being preferred. More selective enzymes that degrade DNA (DNases) or RNA (RNases) can be utilized as well.

### Buffers

Once extracted, buffers will often be utilized to preserve the integrity of protein samples. Buffers are aqueous composed of a weak acid (proton donor) and its conjugate base (proton acceptor). The acid or base is partially neutralized and shows little pH change in response to the addition of stronger acids or bases because of the buffers ability to “absorb” hydrogen ions, which determine pH. The most effective pH range for a buffer is generally one pH unit and is centered around the pKₐ of the system.

In choosing an appropriate buffer system, one generally takes into account the following considerations. (1) The pKₐ of the buffer should be near the desired midpoint pH of the solution. (2) The capacity of the buffer should fall within one to two pH units above or below the desired pH values. If the pH is expected to drop during the procedure, choose a buffer with a pKₐ slightly lower than the midpoint pH. Similarly, if the pH is expected to rise, choose a buffer with a slightly elevated pH. (3) The concentration of the buffer should be adjusted to have enough capacity for the experimental system. (4) The pH of the buffer should be checked at the temperature and concentration which will be used in the experimental system. (5) No more than 50% of the buffer components should be dissociated or neutralized by ionic constituents which are generated within or added to the solution. (6) Buffer materials should not absorb light between the wavelengths of 240-700 nm.

Useful buffers include ADA (Na salt), BES, ethyl glycinate, glycine, PBS, lithium citrate, PIPPS, potassium phosphate (mono- or dibasic), sodium citrate, sodium phosphate, TAPS, Tris base, Tris-HCl, MES, Bis-Tris, PIPES (Na salt), ACES, MOPES, TES, HEPES (Na salt), HEPPS, Tricine, Bicine, CHES, CAPS, MOPSO, DIPSO, HEPPSO, POPSO, AMPSO and CAPSO. Particularly useful buffers for mass spectrometry are volatile buffers, including ammonium bicarbonate and ammonium acetate.

### Mass Spectrometry

A “mass spectrometer” is an analytical instrument that can be used to determine the molecular weights of various substances, such as proteins and nucleic acids. It can also be used in some applications, e.g., to determine the sequence of protein molecules and the chemical composition of virtually any material. Typically, a mass spectrometer comprises four parts: a sample inlet, an ionization source, a mass analyzer, and a detector. A sample is optionally introduced via various types of inlets, e.g., solid probe, GC, or LC, in gas, liquid, or solid phase. The sample is then typically ionized in the ionization source to form one or more ions. The resulting ions are introduced into and manipulated by the mass analyzer. Surviving ions are detected based on mass to charge ratio. In one embodiment, the mass spectrometer bombards the substance under investigation with an electron beam and quantitatively records the result as a spectrum of positive and negative ion fragments. Separation of the ion fragments is on the basis of mass to charge ratio of the ions. If all the ions are singly charged, this separation is essentially based on mass. Traditional quantitative MS has used electrospray ionization (ESI) followed by tandem MS (MS/MS) while newer quantitative methods are being developed using matrix assisted laser desorption/ionization (MALDI) followed by time of flight (TOF) MS.

### A. ESI

ESI is a convenient ionization technique developed by Fenn and colleagues (Fenn et al., Science, 246(4926):64-71, 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice. Kabarle et al., Anal. Chem. 65(20):972A-986A (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer, is delivered to tip through an internal passage from a suitable source (such as from a chromatograph or directly from a sample solution via a liquid flow controller). By applying pressure to the sample in the capillary, the liquid leaves the capillary tip as small highly electrically charged droplets and further undergoes desolvation and breakdown to form single or multicharged gas phase ions in the form of an ion beam. The ions are then collected by the grounded (or negatively charged) interface plate and led through an orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; 6,756,586, 5,572,023 and 5,986,258.

### B. ESI/MS/MS

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., Anal. Chem., 74:2446, 2000) and bioactive peptides (Desiderio et al., Biopolymers, 40:257, 1996). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide. Bucknall et al., J. Am. Soc. Mass Spectrometry, 13(9):1015-27 (2002). Protein quantification has been achieved by quantifying tryptic peptides. Mirgorodskaya et al., Rapid Commun. Mass Spectrom., 14:1226, 2000. Complex mixtures such as crude extracts can be analyzed, but in some instances sample clean up is required. Gobom et al., Anal. Chem. 72:3320, 2000. Desporption electrospray is a new associated technique for sample surface analysis.

### C. SIMS

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

### D. LD-MS and LDLPMS

Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and the separation of fragments is due to different size causing different velocity. Each ion mass will thus have a different flight-time to the detector.

One can either form positive ions or negative ions for analysis. Positive ions are made from regular direct photoionization, but negative ion formation requires a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of negative ion spectra.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

### E. MALDI-TOF-MS

Since its inception and commercial availability, the versatility of MALDI-TOF-MS has been demonstrated convincingly by its extensive use for qualitative analysis. For example, MALDI-TOF-MS has been employed for the characterization of synthetic polymers, peptide and protein analysis (Zaluzec et al., Protein Expr. Purif., 6:109, 1995; Roepstorff et al., EXS, 88:81, 2000), DNA and oligonucleotide sequencing, and the characterization of recombinant proteins. Recently, applications of MALDI-TOF-MS have been extended to include the direct analysis of biological tissues and single cell organisms with the aim of characterizing endogenous peptide and protein constituents. Li et al., Trends Biotechnol., 18:151 (2000); Caprioli et al., Anal. Chem., 69:4751 (1997).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of polypeptides (i.e., peptides and proteins) is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use. Wang et al., J. Agric. Food. Chem., 48:3330 (2000); Desiderio et al., Biopolymers, 40:257 (1996). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products, whereas other studies have demonstrated the potential of MALDI-TOF-MS for the quantification of biologically relevant analytes such as neuropeptides, proteins, antibiotics, or various metabolites in biological tissue or fluid. Muddiman et al., Fres. J. Anal. Chem., 354:103 (1996); Nelson et al., Anal. Chem., 66:1408 (1994). In earlier work it was shown that linear calibration curves could be generated by MALDI-TOF-MS provided that an appropriate internal standard was employed. Duncan et al., Rapid Commun. Mass Spectrom., 7:1090 (1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., J. Mass. Spectrom., 32:1111, 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are important. Only a select group of compounds is useful for the selective desorption of proteins and polypeptides. A review of all the matrix materials available for peptides and proteins shows that there are certain characteristics the compounds must share to be analytically useful. With a few exceptions, most of the matrix materials used are solid organic acids. Liquid matrices have also been investigated, but are not used routinely.

### Automation and High-Throughput Analysis

In some embodiments of the invention, one or more steps of the method can be automated and implemented for the analysis of a very large number of samples. For example, the step of depositing one or more portions of a hydrogel mixture can be done robotically. Likewise, removing the digested mixture and extracting the polypeptides can be done robotically. Commercially available mass spectrometry system (e.g., MALDI instruments) can record the mass spectra of all the extract samples in quick succession. The analysis of the data can also be automated by employing a computer program to analyze generated data. With sufficient automation, a single person, with access to a MALDI instrument could use the automated techniques to measure as many as 1000 samples per day.

In some embodiments, the one or more hydrogel portions are placed in a regular column and row pattern (e.g., corresponding to that found in a standard 96 well plate) in a highly automated fashion, thereby ensuring that the rate of screening is dependent only on the speed of sequential analysis of the mass spectrometer. An automatic sampler can be used to transport samples between the purification system (which includes extraction and/or column purification) and the mass spectrometer. Autosamplers can be purchased from standard laboratory equipment suppliers such as Gilson and CTC Analytics. Such samplers function at rates of about 10 seconds/sample to about 1 min/sample. In some embodiments, the invention also includes a computer and software operably coupled to the apparatus for recording and analyzing mass spectrometer data and for controlling the automatic sampler.

In some embodiments, the method of analyzing polypeptide content is a high-throughput method. “High throughput mass spectrometry” is used herein to refer to a mass spectrometry system that is capable of analyzing samples at a rate of from about 100 or 200 samples per day to about 15,000 samples per day. In general, mass spectrometry and MALDI-MS in particular have proven to be highly amenable to high throughput applications in both clinical and basic research settings. For example, Sequenom Inc. (San Diego, Calif.) has established MALDI-MS as an effective technique in the field of genotype profiling, and is providing diagnostic products in this area. In some embodiments, the method is capable of analyzing about 200 samples in less than an hour, e.g., 200 samples are injected into a mass spectrometer and analyzed in less than an hour. High throughput screening preferably takes advantage of the ability to automate the data acquisition and data analysis methods.

### Applications

Alterations in proteins abundance, structure, or function, act as useful indicators of pathological abnormalities prior to development of clinical symptoms and as such are often useful diagnostic and prognostic biomarkers. The analysis of polypeptide content of animal tissue can be used for various different purposes. Hanash, S., Nature 422, 226-232 (2003). For example, by examining the proteome of various tissues, one can identify subjects that have or are at risk of disease, including infections, cancer, autoimmune disorders, diabetes, or virtually any other condition for which protein aberrations are known. In many cases, the underlying mechanisms of diseases such as cancer are quite complicated in that multiple dysregulated proteins are involved. In other embodiments, analysis of polypeptide content of animal tissue can be used in drug development to identify regulated targets and evaluate drug effects.

In one aspect, the present invention involves the use of mass spectroscopy to diagnosis or predict conditions or disease states in a subject. Ideally, the use of the present invention permits replicate sampling to ensure accuracy, but also permits testing for multiple targets in discrete but spatially related portions of a tissue. Tissue samples may be obtained using protocols described herein.

Conditions that may be diagnosed according to the present invention include, but are not limited to, cancer, infection, congenital disease, exposure to toxicity, and diabetes. Generally, the protein expression of one or more protein targets in the tissue sample will be compared in a standard or known expression level, array or distribution. Alternatively, known healthy tissue may be interrogated in parallel to provide the “normal control” to which the sample is compared.

In another embodiment, the present invention permits the monitoring of disease development, disease progression, or the effects of a treatment on a subject. Such an assay will comprise, essentially, the same steps a diagnostic method with the exception that the timing of the examination will be based on (a) a previous negative diagnostic result, (b) a previous positive diagnostic result, or (c) a prior treatment application.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Localized In-Situ Hydrogel-Mediated Protein Digestion and Extraction Technique for on-Tissue Analysis

Identification of imaged small molecules (e.g. pharmaceuticals, peptides and lipids) commonly proceeds with on-tissue tandem MS (MS/MS) and accurate mass measurements. These experiments usually follow an initial IMS experiment and thus are carried out on the section already imaged or on an adjacent serial tissue specimen. This allows for the best co-localization of the initial imaged precursor ion to the confident MS/MS identification from product ions. However, on-tissue protein identification can be laborious. One approach utilizes traditional proteomic methodologies to identify proteins. Franck et al., Mol. Cell. Prot. 8:2023-2033 (2009). The first step may involve one of several approaches: microextraction from the tissue surface with aqueous/organic solvents, tissue homogenization from multiple neighboring tissue specimens, laser capture microdissection (LCM) collection of regions of interest, or bulk tissue homogenization depending on the localization and predicted relative amount of the desired protein. High-performance liquid chromatography (HPLC) fractionation of this bulk tissue homogenization often follows where the fractions are collected throughout the gradient. Aliquots of these fractions are spotted onto a MALDI target for MS analysis to determine what fractions contain the proteins of interest. The aliquot may be subjected to additional offline clean-up and separations such as sodium dodecyl sulfate polyacrylamide gel electrophoresis (SDS-PAGE) to further isolate the approximate molecular weight fraction in a gel band. A standard in-gel proteolytic digestion procedure is performed, and the product peptides are identified via HPLC-MS/MS and database searching. This process works well for many soluble and more abundant protein species and in applications where there is a relatively large amount of tissue available. However, the process is time consuming, sample (tissue) quantities are often limited and isolating distinct micro-regions of tissue for spatially-directed analysis can be difficult.

A second approach for on-tissue protein identification is the use of in-situ proteolytic digestion. Groseclose, J. Mass Spectrom. 42:254-262 (2007). Typically, serial sections of tissue are employed. On one tissue specimen, the IMS experiment is performed targeting proteins, peptides, lipids or other classes of molecules. On an adjacent serial tissue specimen a proteolytic enzyme (e.g. trypsin) is deposited in a similar array to that of the imaged section. After the enzyme is applied and digestion is allowed to proceed, application of a MALDI matrix follows for peptide imaging. The protein and peptide images are correlated using post-processing tools to direct subsequent on-tissue MS/MS analyses. This approach correlates the original ion image with protein identification through peptide analysis at identical locations on the sections. A drawback of this approach includes the poor on-tissue digestion efficiency caused from rapid drying of the small droplets of enzyme solution that are required to achieve high spatial resolution. Increasing the aqueous content of the enzyme solution and/or spraying/depositing larger droplets can mitigate the problem, but this may lead to delocalization of endogenous biological molecules which will make image correlation and confident identification challenging. Additionally, the total number of tryptic peptides relating to the desired protein species may be limited, reducing the confidence of identification. Previously reported methods for in-situ digestion on tissue specimens do not typically probe as deeply into the proteome when compared to bulk homogenates analyzed by HPLC-MS/MS identification. Yao et al., Proteom. 8:3692-3701 (2008).

The present example describes a spatially-directed simultaneous on-tissue proteolytic digestion and extraction technique to be used in conjunction with existing MALDI IMS workflows. See Harris et al., Anal. Chem. 85, 2717-23 (2013). This approach utilizes on-tissue protein identification within a hydrogel microreactor network to simultaneously digest and extract proteins and peptides followed by traditional peptide sequence analysis.

### EXPERIMENTAL

### Reagents

For hydrogel synthesis, alginic acid sodium salt was purchased from Alfa Aesar (Ward Hill, Mass. USA) and calcium chloride dihydrate was purchased from J. T. Baker (Center Valley, Pa. USA). The hydrogel additives Triton X-100, ammonium bicarbonate and proteomics grade trypsin from porcine pancreas (dimethylated), the MALDI matrices 2,5-dihydroxybenzoic acid (DHB, 98%) and sinapinic acid (98%) and the acids trifluoroacetic and formic acid and were all purchased from Sigma Aldrich (St. Louis, Mo. USA). Solvents (ethanol, xylenes, methanol and acetonitrile) were all HPLC grade and the histological dyes (hematoxylin and eosin) were purchased from Fisher Scientific (Fairlawn, N.J. USA). 18 MΩ water was provided via a Millipore Milli-Q Synthesis A10 (Billerica, Mass. USA). All reagent listed were used without additional purification.

### Hydrogel Fabrication

Hydrogels were fabricated using a previously described method utilizing template chromatography paper (Whatman, Buckinghamshire, UK). Bracher et al., ACS App. Mat. Inter. 1:1807-1812 (2009); Bracher et al., Adv. Mat. 21:445-450 (2009); Bracher et al., Soft Mat. 6:4303-4309 (2010). Briefly, designs of various shapes and sizes were created in computer software and color laser printed (Bizhub C360, Konica Minolta, Ramsey, N.J. USA) onto the chromatography paper (20×20 cm). The color levels were increased to maximize the amount of ink printed. The paper was reprinted with an identical pattern for a total of 3 times to ensure a thick ink coating. The paper template was then cut out and heated for 120 sec on each side with a heat gun (low setting ˜300° C., 1400 W Milwaukee heat gun, Brookfield, Wis. USA). This allowed for the hydrophobic color ink to melt and penetrate into the paper for defined hydrophobic boundaries. The patterned templates were then soaked in 500 mM CaCl₂ for 60 sec as the free Ca²⁺ ions act as a crosslinking agent to penetrate into the applied polymer creating an ionotropic hydrogel. The hydrogel polymer (alginate) was diluted to 2% w/v in water. The polymer solution was mixed with 200 mM ammonium bicarbonate with 0.02% Triton X-100 in a 1:1 ratio such that the final polymer solution was 1% w/v in 100 mM ammonium bicarbonate and 0.01% Triton X-100. This solution was first mixed with trypsin prior to being spotted onto the calcium soaked pattern templates. The volume deposited (Vₜₒₜ) and the amounts of trypsin in each digestion are outlined per experiment later herein.

### Tissue Specimening and Pre-Treatment

Brains from Sprague-Dawley rats were collected and stored at −80° C. prior to sectioning (Pel-Freez Biologicals, Rogers, Ark. USA). Coronal and sagittal sections (12 and 9 μm, respectively) were taken at −19° C. on a cryostat (Leica Microsystems Inc., Bannockburn, Ill. USA). Tissue specimens were thaw mounted onto premium microscope slides (Fisher Scientific, Fairlawn, N.J. USA) which were cleaned previously in 100% ethanol (twice) and 100% methanol rinses (60 sec each).

### Protein Digestion, Extraction and Identification

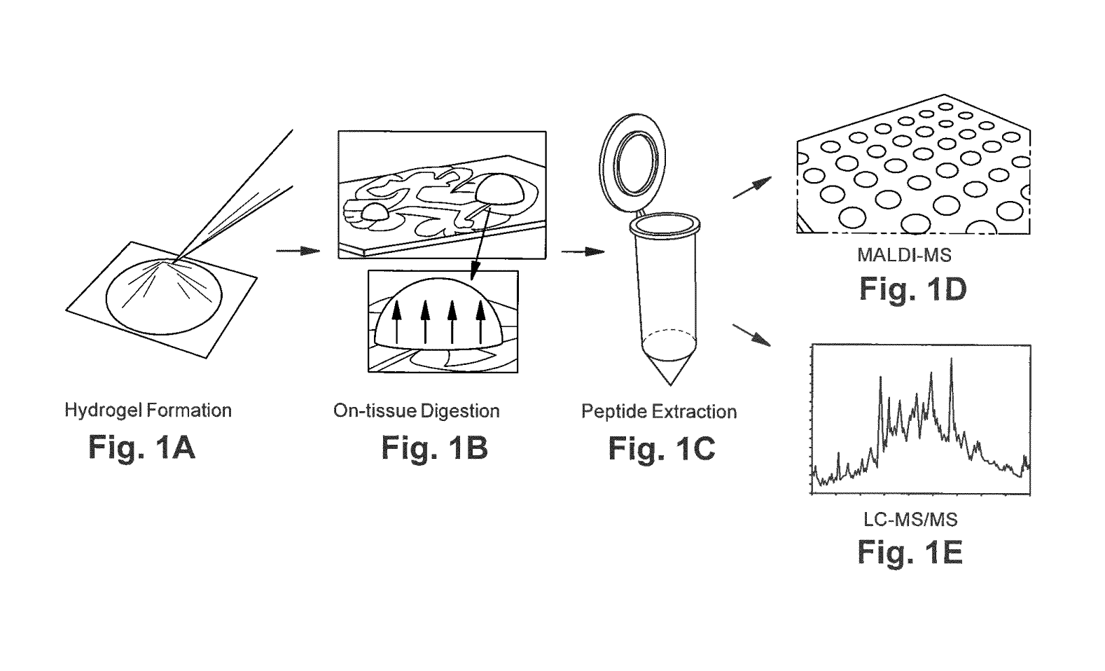

The ionotropic hydrogel mixture was spotted onto the paper target where it was polymerized (<5 minutes) (FIG. 1a). The gel was then removed from the paper pattern via a spatula, thin nosed tweezers or suction via a pipette tip and placed onto a mounted piece of tissue (FIG. 1b). To retain the enzyme activity and improve digest efficiency, the slide was placed in a glass petri dish with a small (4 cm×4 cm) wetted (250-1000 μL depending on gel and tissue sizes to minimize condensation on tissue) piece of paper towel underneath the slide. The petri dish was closed with the glass lid and wrapped with parafilm to make an air-tight seal. The petri dish was placed in an oven and heated to a desired temperature (e.g. 50° C.) for 6-12 hours depending on the tissue thickness, size/shape of the gel, enzyme, etc. This process ensured that the hydrogel did not become dry during digestion. After the digestion, the gel was removed and placed in a micro-centrifuge tube to undergo organic (50% acetonitrile/5% formic acid) and aqueous (50 mM NH₄HCO₃, 25 mM CaCl₂) solvent extractions (process repeated twice) to shrink and swell the hydrogel to extract digested peptides (FIG. 1c). The supernatants of each extraction are combined and dried in a centrifugal vacuum concentrator (SPD Speedvac, Thermo Scientific, Waltham, Mass. USA). The reconstituted extract (50-75 μL in 0.1% formic acid) was then spotted for MALDI MS (FIG. 1d) and LC-MS/MS (FIG. 1e) and could be stored at −80° C. All microscopy was performed with an Olympus BX 50 (Center Valley, Pa. USA) with the following histology procedure: 95% ethanol 30 sec, purified water 30 sec, hematoxylin 120 sec, water 15 sec, 70% ethanol 15 sec, 95% ethanol 15 sec, eosin 60 sec, 95% ethanol 15 sec, 100% ethanol 15 sec, xylenes 120 sec.

### Mass Spectrometry Analysis

MALDI MS profiling was conducted by spotting 2 μL of a 1:1 mix of reconstituted digest mix with 20 mg mL⁻¹ sinapinic acid in 90% acetonitrile and 0.25% trifluoroacetic acid. A Bruker Ultraflextreme™ mass spectrometer (Bremen, Germany) was used to acquire spectra with the spot size set to medium, laser energy at 78% and laser frequency set to 500 Hz. Resulting spectra were an average of multiple laser shots. This data was processed using FlexAnalysis v3.3.

MALDI IMS experiments were performed with a Waters Synapt™ G2 instrument (Manchester, UK) at laser frequency 1000 Hz, 1 scan sec⁻¹ and 125 μm×125 μm laser step. MS acquisition was in Resolution mode with trap and transfer collision energies set to 4 V and 2 V, respectively. Traveling wave ion mobility settings were as follows: nitrogen gas flow at 65 mL min⁻¹ (2.5 mbar), helium cell gas flow at 165 mL min⁻¹, 450 μs pulse delay, ion mobility wave height 40 V, variable ion mobility wave velocity starting at 1100 m s⁻¹ and ending 400 m s⁻¹ with the velocity ramped over 100% of the cycle. DHB was applied with an automated sprayer at a concentration of 5 mg mL⁻¹ in 90% acetonitrile and 0.25% trifluoroacetic acid for a total of 4 passes (HTX Technologies TM Sprayer, Carrboro, N.C. USA). On-tissue MS/MS was performed at designated collision energies (32.5 V for PC 36:1, 35 V for PC 34:1 and 37.5 V for PC 36:4) applied in the trap triwave after precursor ion selection in the quadrupole. Data was processed in Waters HD Imaging software (images normalized to total ion current), Driftscope v2.2 and Masslynx.

Extracted peptides were analyzed by a 75 min data dependent LC-MS/MS analysis. Briefly, peptides were loaded via pressure cell onto a 40 mm by 0.1 mm self-packed reversed phase (Jupiter 5 μM, 300 Å C₁₈—Phenomenex, Torrance, Calif. USA) trapping column fritted into an M520 filter union (IDEX, Lake Forest, Ill. USA). After loading and equilibration, this trapping column was attached to a 200 mm by 0.1 mm (Jupiter 3 μm, 300 Å C₁₈—Phenomenex, Torrance, Calif. USA), self-packed analytical column with a laser pulled tip (i.d. 1 μm) coupled directly to an LTQ linear ion trap (Thermo Scientific, Waltham, Mass. USA) using a nanoelectrospray source. Reverse phase separation was performed on a Waters nanoAcquity™ UPLC (Milford, Mass. USA) using the following gradient run at 500 nL min⁻¹ flow rate: initial flow of 98% A (0.1% formic acid), 2% B (acetonitrile, 0.1% formic acid) ramped to 25% B over 45 min, to 90% B at 60 min where it remained for 5 min, and then a 2 min ramp back to 2% B where it remained for an additional 8 min. A series of full scan mass spectra followed by 5 data-dependent MS/MS spectra was collected throughout the run and dynamic exclusion was enabled to minimize acquisition of redundant spectra. MS/MS spectra were searched via SEQUEST against a rat database (UniprotKB taxon 10116—reference proteome set) that also contained reversed versions for each of the entries. Yates et al., Anal. Chem. 67:1426-1436 (1995). Identifications were filtered and collated at the protein level using Scaffold (Proteome Software, Portland, Oreg. USA) with a 0.5% false discovery recovery rate.

### Results and Discussion

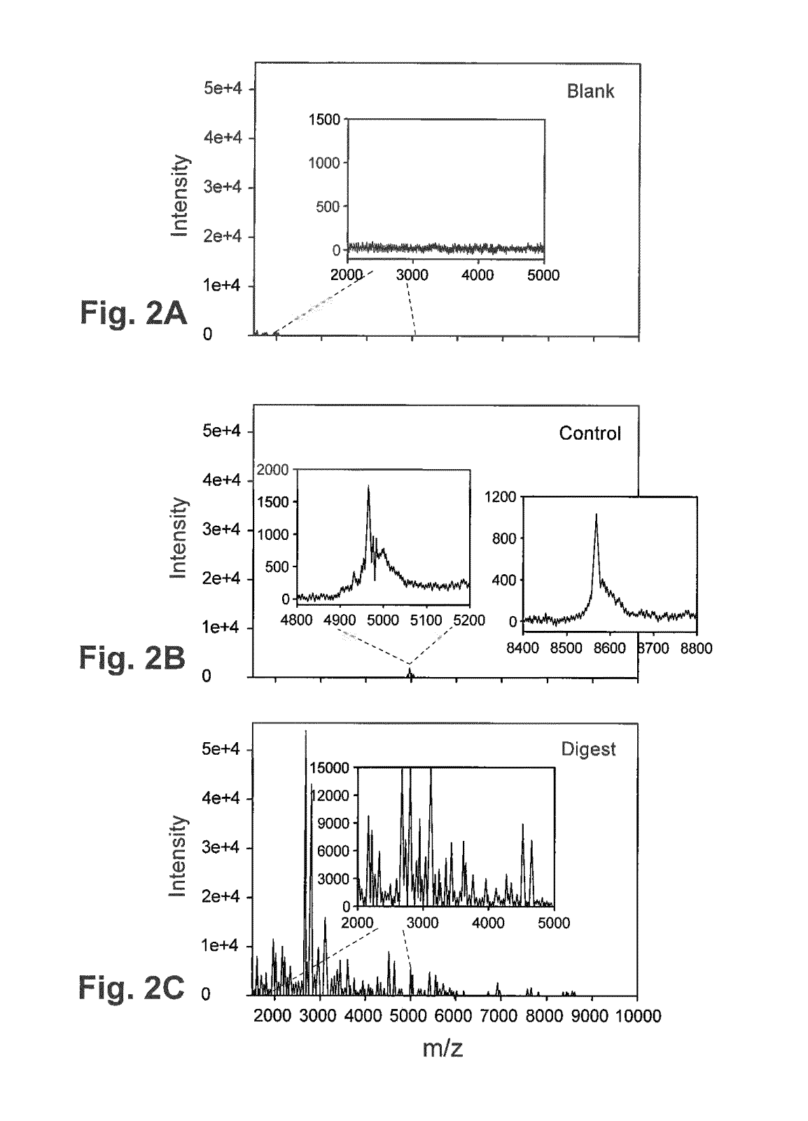

A workflow for a typical hydrogel mediated on-tissue digestion is shown in FIG. 1. A circular 4 mm hydrogel containing trypsin (Vₜₒₜ=18 μL, 20 μg trypsin in 100 mM NH₄HCO₃) was placed on a 12 μm thick section of rat cerebrum and allowed to react for 6 hours in a humidity chamber at 50° C. Following digestion, the gel underwent solvent extraction for peptide recovery, and the resulting peptides were dried down and reconstituted to a volume of 75 L. MALDI MS spectra (2000 shots summed, linear mode) of a blank (trypsin loaded off-tissue, FIG. 2a), control (no trypsin on-tissue, FIG. 2b) and digested (trypsin loaded on-tissue, FIG. 2c) hydrogels obtained from 2% (by volume) of the total extract. Abundant signals were present throughout the mass range demonstrating extensive on-tissue digestion and efficient extraction from the hydrogel network of the trypsin loaded on-tissue hydrogel (FIG. 2c). The absence of signals from the hydrogel polymer is the result of the presence of CaCl₂ in the extraction buffer which maintains the integrity of the ionotropic hydrogel, and prevents contamination of the supernatant. For a blank, a hydrogel with trypsin was placed on an empty microscope slide (FIG. 2a) and as a control, a gel without trypsin was placed on a serial section of tissue (FIG. 2b). As shown in the blank spectrum (FIG. 2a), no signal is present indicating autolytic trypsin peptides are below the detection limit for the amount of trypsin used in this study. The control spectrum shows a lack of peptide signals in the lower m/z 1500-4000 range, but some low intensity signals in the higher mass range (m/z 4500-9000) were observed, e.g. m/z 4964 and 8566 (FIG. 2b inserts). It is likely that these signals are from the abundant and commonly observed intact proteins thymosin β4 and ubiquitin, respectively. Rahman et al., Can. Res. 71:3009-3017 (2011). Interestingly, the presence of these ions indicates that abundant smaller proteins can passively diffuse into these gels as well. Potential modifications of the procedure to improve the extraction of intact proteins into the gels include increasing the porosity (Wu et al., Chem. Rev. 112:3959-4015 (2012)) or altering the alginate (or other hydrogel) properties. Pawar S N, Edgar K J., Biomat, 33:3279-3305 (2012). This could be of particular usefulness in off-line solution digestions of hydrogel extracted proteins and top-down protein identification workflows.

The LC-MS/MS analysis of this sample digestion identified 211 proteins. This amount is in range with similarly sized punched sections of mouse liver fresh frozen and formalin-fixed and paraffin-embedded tissues analyzed by LC MALDI FTICR and TOF/TOF analysis (Aemi et al., Anal. Chem. 81:7490-7495 (2009)), and comparable to simple protein extractions without detergent additives from bulk homogenization of mouse brains analyzed by LC-MS/MS. Shevchenko et al., J. Prot. Res. 11:2441-2451 (2012). There were 85 proteins with extensive sequence coverage, having more than 5 unique peptides identified. Also of interest were high molecular weight proteins that are not readily observed in typical MALDI IMS experiments (Table 1). For example, 26 large proteins were confidently identified with predicted molecular weights greater than 100 kDa. Many of these higher molecular weight proteins were identified as membrane proteins (e.g. several Na+/K+ transporting ATPases, neurofascin, clathrin heavy chain and ankyrin-2), cytoskeletal proteins (e.g. a actinin, microtubule-associated proteins and spectrin), extracellular matrix proteins (restrictin) and cytoplasmic proteins (e.g. puromycin-sensitive aminopeptidase, protein bassoon and dynein heavy chain) Another 77 of the 211 proteins were detected in the 50-99 kDa range, also a challenging intermediate high mass range for MALDI IMS.

TABLE 1
High molecular weight (>100 kDa) proteins identified
with on-tissue hydrogel-mediated protein digestion and
extraction on rat brain cerebrum.

| | UniProt | Molecular | # | # |
|---|---|---|---|---|
| | Accession | Weight | Unique | Assigned |
| Protein Name | Number | (kDa) | Peptides | Spectra |
| Hexokinase type 1 | P05708 | 102 | 4 | 6 |
| α actinin 1 | Q6GMN8 | 103 | 5 | 10 |
| Puromycin-sensitive | P55786* | 103 | 4 | 6 |
| aminopeptidase | | | | |
| Type II brain 4.1 minor | Q9JMB2 | 107 | 3 | 5 |
| isoform | | | | |
| Na⁺/K⁺ ATPase α3 subunit | P06687 | 112 | 20 | 67 |
| Na⁺/K⁺ transporting | D3ZSA3 | 112 | 3 | 6 |
| ATPase α2 chain precursor | | | | |
| Na⁺/K⁺ transporting | P06685 | 113 | 10 | 17 |
| ATPase α1 chain precursor | | | | |
| Contactin-1 precursor | Q63198 | 113 | 6 | 9 |
| Neurofilament heavy | F1LRZ7 | 114 | 8 | 11 |
| polypeptide | | | | |
| Ubiquitin-activating | Q5U300 | 118 | 3 | 4 |
| enzyme E1 | | | | |
| Electroneutral K⁺/Cl⁻ | Q63633 | 124 | 2 | 2 |
| cotransporter 2 | | | | |
| Plasma membrane | P11506 | 137 | 6 | 9 |
| Ca₂⁺ ATPase isoform 2 | | | | |
| Neurofascin | P97685 | 138 | 2 | 2 |
| Plasma membrane Ca₂⁺ | P11505 | 139 | 3 | 4 |
| transporting ATPase 1 | | | | |
| Ras GTPase-activating | Q9QUH6 | 143 | 3 | 3 |
| protein SynGAP | | | | |
| Tenascin-R precursor | Q92752* | 150 | 2 | 3 |
| (Restrictin) | | | | |
| Clathrin heavy chain 1 | P11442 | 191 | 9 | 13 |
| Microtubule-associated | P15146 | 199 | 21 | 46 |
| protein 2 | | | | |
| Microtubule-associated | P15205 | 270 | 8 | 13 |
| protein 1B | | | | |
| Spectrin β chain, brain 2 | Q9QWN8 | 271 | 5 | 7 |
| Non-erythroid spectrin β | Q6XD99 | 274 | 29 | 47 |
| Spectrin α chain, brain | P16086 | 285 | 60 | 109 |
| Microtubule-associated | G3V7U2 | 300 | 15 | 27 |
| protein 1A | | | | |
| Protein bassoon | O88778 | 418 | 2 | 4 |
| Ankyrin-2 | Q01484* | 437 | 5 | 5 |
| Dynein heavy chain, | P38650 | 532 | 8 | 9 |
| cytosolic | | | | |

*Predicted protein based on sequence homology from Homo sapiens

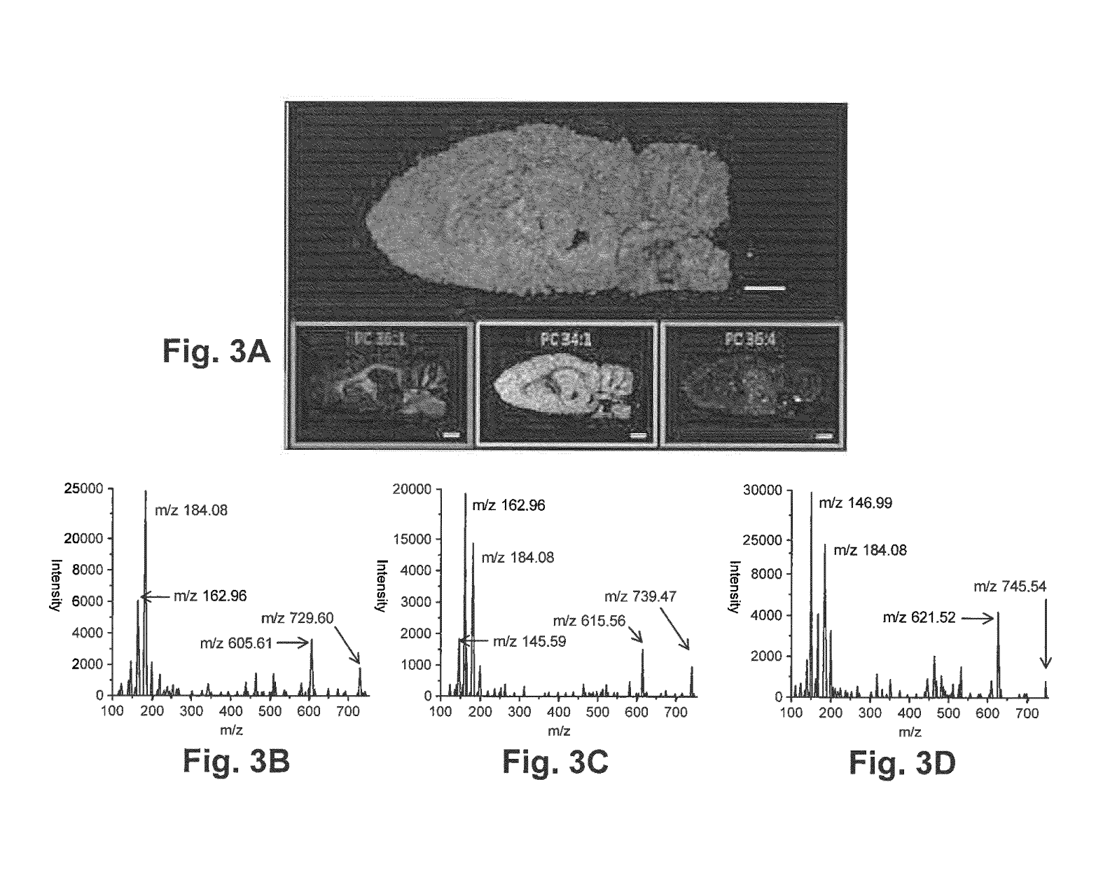

The hydrogels can also be used for a sequential analysis workflow in conjunction with a previously imaged section of tissue. In this case, the digestion and extraction occur after the initial IMS and on-tissue MS/MS, but before histological staining (FIG. 3). A 9 μm thick section of rat brain was imaged with the traveling wave ion mobility spectrometry (TWIMS) separation. The use of TWIMS in MALDI IMS experiments separates MALDI matrix ions from the targeted biological ions. McLean et al., J. Mass Spectrom. 42:1099-1105 (2007); Djidja et al., J. Prot. Res. 8:4876-4884 (2009). Shown in FIG. 3a is an overlaid ion image of three identified lipid species assigned to phosphatidylcholines (PC) at m/z 788.62, 798.54 and 804.55, relating to PC 36:1, PC 34:1 and PC 36:4, respectively. These species were identified based on accurate mass and MS/MS (FIG. 3b-d) of neighboring pixels after the initial full scan IMS experiment, and confirmation that these ions fell into the lipid trend line in the ion mobility dimension of separation.

After the lipid IMS and MS/MS experiments were completed, the tissue was not visibly damaged since the laser energy used (energy level 275, approximately 46% power) was sufficiently high enough to obtain signal, but below the threshold for ablation of the tissue. Before proceeding to the in-situ digestion, the tissue was washed two times for 30 sec (70% ethanol and 100% ethanol, respectively) to remove the residual DHB matrix, salts and lipids on the tissue surface. A hydrogel disc (diameter=1.5 mm, Vₜₒₜ=8 μL containing 10 μg trypsin and 0.1% Triton X-100 in 100 mM NH₄HCO₃) was placed on the cerebellum region of the tissue and incubated for 6 hours at 50° C. For comparison, another imaged rat brain tissue specimen was washed and a 1.5 mm (diameter) region of cerebellum was removed for an in-solution digestion (Vₛₒₗ=250 μL) with the same amount of trypsin and Triton X-100. A third imaged tissue specimen was treated the same as the previous two, but a hydrogel without trypsin was placed on the cerebellum as a control. After 6 hours of incubation, the control gel was placed in a 250 μL solution with 10 μg trypsin and 0.1% Triton X-100 in 100 mM NH₄HCO₃ for 6 hours at 50° C. All samples were dried and reconstituted to identical volumes (V=50 μL), and the samples were analyzed via LC-MS/MS.

The on-tissue digestion using the hydrogel protocol identified 96 proteins compared to 147 for the in-solution tissue digestion and 22 proteins for the control hydrogel solution digestion. The identified high molecular weight (>100 kDa) proteins again reveals many membrane (e.g. hexokinase-1, several Na+/K+ transporting ATPases, neural cell adhesion molecule 1, etc.) and cytoskeletal proteins (e.g. microtubule-associated proteins, neurofilament heavy polypeptide, spectrin, etc.) identified using the homogenization and hydrogel digestions (Table 2). However, the digestion efficiencies of the two approaches differed. Based on the number of unique peptides identified and the number of assigned spectra, the in-solution homogenization digestion was a more extensive, albeit destructive, digestion procedure compared to the current iteration of the hydrogel digestion. However, given that the same amount of trypsin was used in both methods, the difference may be attributed to the limited volume of the hydrogel used on-tissue (8 μL) that was more than 30×smaller compared to the solution volume of the homogenization (250 μL), and the requirement for proteins and peptides to migrate into the hydrogel during the digestion incubation. The accessibility of proteins for digestion was greater in the homogenization procedure since trypsin mobility in solution is greater than in the hydrogel network, and peptides are released into solution rather than required to diffuse from tissue into the hydrogels. Future iterations of on-tissue hydrogels can be produced to reduce the polymer network density by using alternative hydrogel compositions and creating gels with alternative geometries to increase the total volume-to-surface area ratio of the structure.

TABLE 2
Comparison of the high molecular weight (>100 kDa) proteins identified
with an in-solution homogenization protocol and an on-tissue hydrogel-mediated
protein digestion on a previously imaged rat brain cerebellum.

| | | | In-solution | | On-tissue | |
|---|---|---|---|---|---|---|
| | | | Homogenization | | Hydrogel | |
| | UniProt | Molecular | # | # | # | # |
| Protein | Accession | Weight | Unique | Assigned | Unique | Assigned |
| Name | Number | (kDa) | Peptides | Spectra | Peptides | Spectra |
| Microtubule-associated | Q63560 | 100 | 6 | 6 | 4 | 4 |
| protein 6 | | | | | | |
| Hexokinase-1 | P05708 | 102 | 3 | 3 | 3 | 4 |
| Na^{+/}K⁺ transporting | P06687 | 112 | 18 | 24 | 8 | 9 |
| ATPase subunit α 3 | | | | | | |
| Na⁺/K⁺ transporting | P06686 | 112 | 7 | 8 | 2 | 2 |
| ATPase subunit α 2 | | | | | | |
| Na⁺/K⁺ transporting | P06685 | 113 | 6 | 7 | 2 | 2 |
| ATPase subunit α 1 | | | | | | |
| Contactin-1 | Q63198 | 113 | 3 | 3 | 3 | 4 |
| Neurofilament heavy | F1LRZ7 | 114 | 7 | 9 | 2 | 2 |
| polypeptide | | | | | | |
| Sarcoplasmic/endoplasmic | P11507 | 115 | 4 | 4 | 2 | 2 |
| reticulum Ca²⁺ ATPase 2 | | | | | | |
| Neural cell adhesion | F1LUV9 | 119 | 5 | 6 | 3 | 3 |
| molecule 1 | | | | | | |
| Plasma membrane Ca²⁺ | P11506 | 137 | 7 | 9 | 7 | 7 |
| transporting ATPase 2 | | | | | | |
| Clathrin heavy chain 1 | D4AD25 | 192 | 14 | 16 | 2 | 2 |
| Microtubule-associated | P15146 | 202 | 6 | 6 | 2 | 2 |
| protein 2 | | | | | | |
| Microtubule-associated | F1LRL9 | 270 | 5 | 5 | 3 | 3 |
| protein 1 light chain LC1 | | | | | | |
| Spectrin β 2, isoform | G3V6S0 | 274 | 20 | 20 | 9 | 10 |
| CRA_a | | | | | | |
| Spectrin α chain, brain | E9PU22 | 285 | 38 | 41 | 10 | 11 |
| Microtubule-associated | P34926 | 300 | 4 | 5 | 2 | 2 |
| protein 1A | | | | | | |
| Cytoplasmic dynein 1 | P38650 | 532 | 6 | 6 | 3 | 3 |
| heavy chain 1 | | | | | | |

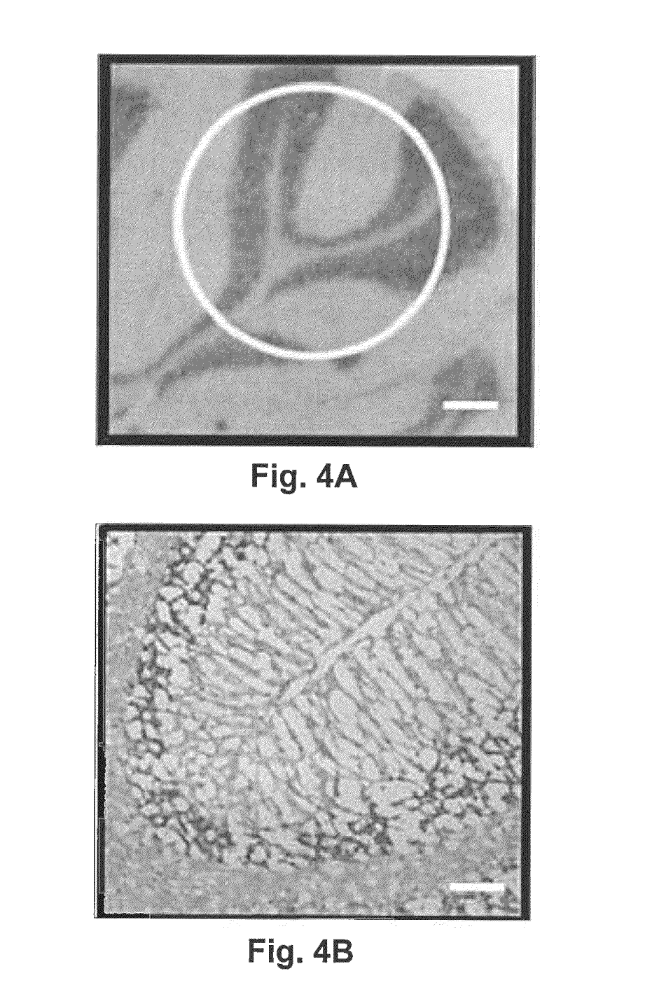

The hydrogel digested tissue specimens can be used for additional analysis such as histological staining (hematoxylin and eosin stain, H & E) for evaluation of cells and surrounding tissue regions. The hydrogel-digested tissue was washed, stained and analyzed by microscopy (FIG. 4). A serial rat brain section that had not been imaged or treated previously was stained with H & E by the identical procedure for comparison purposes (FIG. 4a). It is apparent that there are differences between the two stained tissue specimens mainly in the cellular density. There are a lower number of nuclei (stained blue/purple by hematoxylin) present in the imaged and extracted tissue specimen compared to an untreated section (FIG. 4b). The losses are due to a combination of cellular material being extracted into the hydrogel during incubation/digestion, and the extensive washing steps prior to staining which may have dislodged cellular material from the slide surface. The loss of tissue into the hydrogel is a critical insight into the current function of the gels. Future refinements to these materials will focus on enhancing extraction of already imaged cells and cellular material to maximize proteomic coverage and/or minimizing the observed tissue damage while still obtaining useful proteomic information to be used in conjunction with histological examination of the same tissue specimen. Nonetheless, differential identification of regions (e.g. extent of myelination, white and grey matter regions, location of granular cells) within the tissue was possible in this first study using the protocol for H & E staining. The inventors are developing new staining protocols that are amenable to the pretreatment of the tissue specimens to prevent any potential damage to the tissue specimen.

### CONCLUSION

Hydrogel mediated on-tissue digestion has been demonstrated to be a simple and inexpensive tool to be added to existing IMS methods for protein identification. The hydrogels used in this study are rapidly formed, and can be shaped to match the size of the particular tissue region in question. This example demonstrates that sequential experiments can be performed on a single tissue specimen, i.e. IMS, on-tissue MS/MS, protein identification via LC-MS/MS and histology.

### Example 2

### Histology-Directed Microwave Assisted Enzymatic Protein Digestion for MALDI MS Analysis of Mammalian Tissue

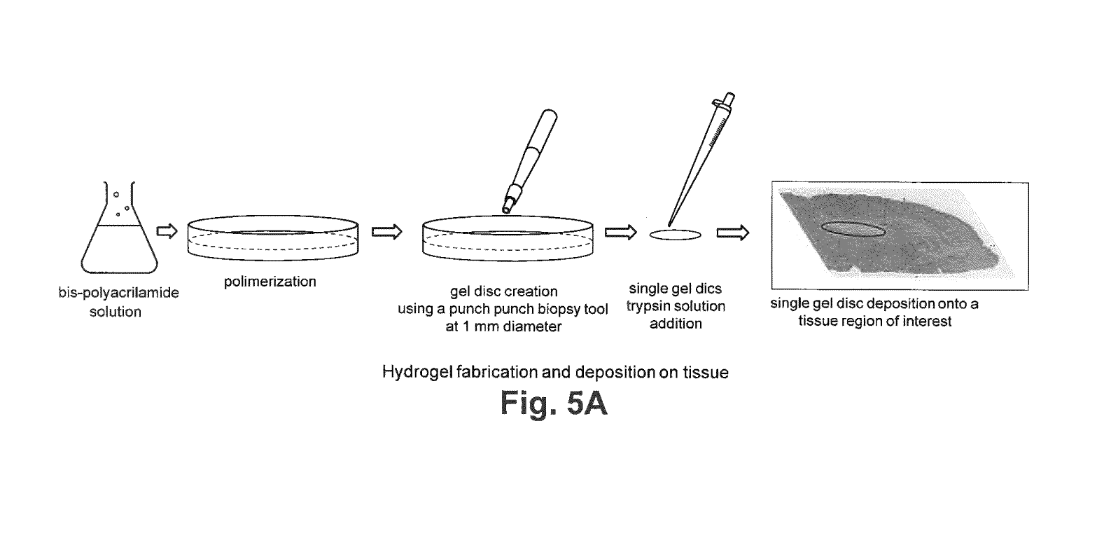

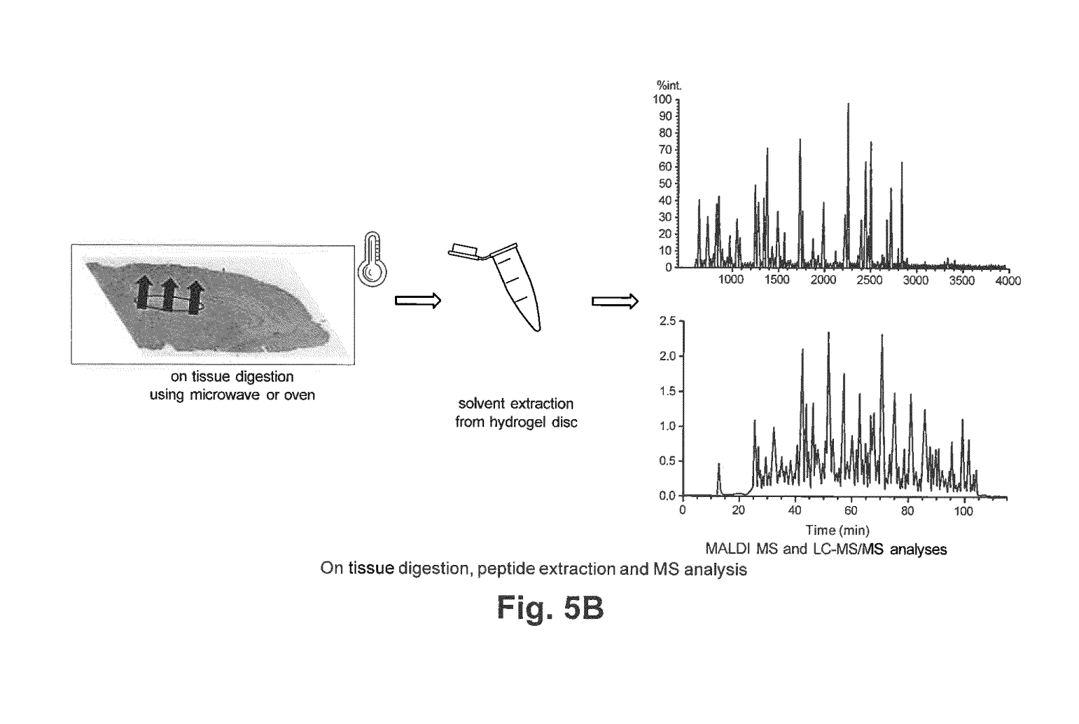

This example presents an on-tissue proteolytic digestion and peptide extraction method using microwave irradiation for in situ analysis of proteins from spatially defined regions of a tissue specimen. The methodology utilizes hydrogel discs (1 mm diameter) imbibed with trypsin solution. The hydrogel discs are applied to a tissue specimen, directing enzymatic digestion to a spatially confined area of the tissue. By incorporating applying microwave radiation, protein digestion takes place is performed on-tissue in 2 minutes on-tissue, and the extracted peptides are then analyzed via MALDI MS and LC-MS/MS. The reliability and reproducibility of the microwave-assisted hydrogel mediated on-tissue digestion was demonstrated by the comparison with other on-tissue digestion strategies, including comparisons with conventional heating and in-solution digestion. All the experiments were replicated and LC-MS/MS data were evaluated considering the number of identified proteins as well as the number of protein groups and of distinct peptides. The results of this study demonstrate that a rapid and reliable protein digestion can be performed on a single thin tissue specimen while preserving the tissue architecture, and the resulting peptides are extracted in sufficient abundance to permit robust analysis using LC-MS/MS. An overview of the workflow involved is shown in FIG. 5. This approach will be most useful for samples that have limited availability but are needed for multiple analyses, especially for the correlation of proteomics results data with histology and immunohistochemistry.

### Materials and Methods

For the hydrogel synthesis acrylamide/bisacrylamide was purchased from Biorad Life Sciences (Hercules, Calif., USA), while ammonium persulfate was purchased from Sigma Aldrich (St. Louis, Mo., USA). The hydrogel additives, ammonium bicarbonate, the MALDI matrix alpha-cyano-4-hydroxycinnamic acid (98%), the acids trifluoroacetic (TFA) and formic (FA) were all purchased from Sigma Aldrich (St. Louis, Mo., USA). Punch biopsies were purchased from Acuderm, Inc. (Ft. Lauderdale, Fla.). Mass spectrometry grade Trypsin Gold was purchased from Promega Corporation (Madison, Wis., USA). HPLC grade solvents (ethanol, xylenes, methanol, and acetonitrile) and histological dyes (hematoxylin and eosin) were purchased from Fisher Scientific (Fairlawn, N.J., USA). Xylene was purchased from Acros (Morris Plains, N.J.). Water was provided via Millipore Milli-Q Synthesis A10 (Billerica, Mass., USA). All reagents listed were used without additional purification.

### Hydrogel Discs Fabrication

Fabrication of 7.5% polyacrylamide hydrogels was carried out following and modifying a previously developed procedure: A volume of 1.24 mL of 30% acrylamide/bisacrylamide solution was added to 1.26 mL TRIS buffer at pH 6.8 and 2.45 mL of water. Nicklay et al., Anal Chem. 85(15):7191-6 (2013). The solution was degassed under vacuum for a minimum of 30 min before adding 50 μL of 10% ammonium persulfate and of 10 μL of TEMED. The solution was mixed by inversion and placed into a small Petri dish to polymerize for 30 min. Finally, punch biopsy tools were used to cut the microwells in a variety of sizes (1, 1.5, 3 mm diameter). Each individual microwell of hydrogel was placed in an eppendorf tube, dried fully in a speedvac and stored at −80° C. until use.

### Tissue Specimening and Pretreatment

Fresh frozen rat brain was purchased from Pel-Freez Biologicals (Rogers, Ariz.) and tissue specimens were prepared at 8 μm thickness using a Leica CM3050 cryostat (Leica Microsystems GmbH, Wetzlar, Germany). Frozen tissue specimens were thaw mounted on microscope slides or placed into eppendorf tubes (for the homogenization procedure) and stored in a desiccator until needed. Each tissue specimen was rinsed using ethanol (95%, 30 sec; 70%, 30 sec) to remove salts, lipids and to obtain optimal sensitivity for MS analysis of the digested extracts. Seeley et al., J. Am. Soc. Mass Spectrom. 19, 1069-77 (2008).

### On-Tissue Microwave Digestion

Hydrogels were re-hydrated for 15 mins using 20 μL of 1 μg/mL trypsin (in 100 mM ammonium bicarbonate) and then placed over the tissue region of interest (brain thalamic region) guided by the histological features on corresponding serial H&E stained tissue specimen. The tissue specimens were incubated in a microwave oven (1.65 kW) for 2 min set at 10% of the power, to accelerate protein digestion. Each hydrogel disc was removed from the tissue specimen and placed in separate eppendorf tubes. Peptides imbibed into the microwell hydrogels were extracted by organic (50% acetonitrile/5% formic acid) and aqueous (100 mM ammonium bicarbonate) solvents, a process that was repeated three times. The supernatant collected from each extraction were combined and dried in a centrifugal vacuum concentrator (SPD Speedvac, Thermo Scientific, Waltham, Mass., USA). The reconstituted extracts (20 μL, 0.1% formic acid) were spotted for MALDI MS analysis and then stored at −20° C. until LC-MS/MS analysis was performed.

Other on-Tissue Digestion Strategies

Further protein digestion experiments were carried out on serially prepared tissue specimens. First, tissue specimens were incubated using a conventional oven at 50° C. for 4 hours: hydrogel discs were still used to allow the digestion to take place on the brain thalamic region. After digestion, peptides were extracted from the gel following the same procedure already described for the microwave digestion. Second, since hydrogels were fabricated at 1 mm diameter, the rat brain biopsy was also punched into the thalamic region using a 1 mm punch biopsy tool. This approach precisely controls the amount of tissue exposed to the hydrogel and allows for the homogenization and digestion of the same amount of tissue using conventional sample preparation methods. Serial sections from the 1 min tissue core were cryosectioned following the same protocol described above. One set of digestion experiments was carried out: n=3 tissue core sections were mounted on microscope slides and hydrogel discs (trypsin embedded) were placed on top and incubated via conventional oven at 50° C. for 4 hours. Second, n=3 tissue core sections were marked using a hydrophobic pen and trypsin was manually spotted and incubated overnight at 37° C. Finally, other serial tissue core sections from the same tissue specimen were placed into separate eppendorf tubes and the homogenized as previously described. Bodzon-Kulakowska et al., J. Chromatogr. B. Analyt. Technol. Biomed. Life Sci. 849, 1-31 (2007). The digestion was conducted at 37° C. overnight, and the digested peptides were extracted following the same procedure described above.

### Mass Spectrometry Analysis and Data Processing

MALDI MS analyses were carried out using an UltrafleXtreme™ MALDI TOF/TOF spectrometer (Broker Daltonics, Billerica, Mass.) equipped with a SmartBeam™ II laser and operating in positive polarity, reflectron mode. Spectra were acquired in the range of m/z 500-4000. Flex Control 3.3 software was used for spectra acquisition. The reproducibility of the hydrogel-based digestion was evaluated using a set of three technical replicates within the same tissue sample at different but histologically identical locations. All spectra were processed using the same preprocessing procedure to ensure overall consistency. Briefly, they were baseline-corrected and normalized according to their total ion current, excluding the top 5% of intensity values to avoid bias by highly abundant species. The Mann-Whitney U test and Kruskal-Wallis test were applied to evaluate statistically significant of differences (protein groups and distinct peptides) between groups (all different digestion strategies). The Mann-Whitney U test and Kruskal-Wallis test are the analogous nonparametric methods of t-test and one-way between-groups of variance (analysis of variance, ANOVA), respectively.

### LC-MS/MS Analysis

Resulting peptides were analyzed by a 70 minute data dependent LC-MS/MS analysis. Briefly, peptides were loaded via pressure cell onto a 40 mm by 0.1 mm self-packed reversed phase (Jupiter 5 um, 300 A—Phenomonex) trapping column fritted into an M520 filter union (IDEX). After loading and equilibration, this trapping column was attached to a 200 mm by 0.1 mm (Jupiter 3 micron, 300 A), self-packed analytical column coupled directly to an LTQ (ThermoFisher) using a nanoelectrospray source. A series of a full scan mass spectrum followed by 5 data-dependent tandem mass spectra (MS/MS) was collected throughout the run, and dynamic exclusion was enabled to minimize acquisition of redundant spectra. MS/MS spectra were searched via SEQUEST against a human database (UniprotKB—reference proteome set) that also contained a reversed version for each of the entries. Yates et al., Anal. Chem. 67, 1426-36 (1995). Identifications were filtered to 2 peptides per protein and 0% peptide false detection rate and collated at the protein level using Scaffold (Proteome Software). Furthermore, IDPicker 3 software used to filter the resulting identifications to a 5% FDR at the peptide level and collate the individual proteins, requiring a minimum of 2 peptides per protein. Holman et al., Curr Protoc Bioinforma. 13, unit 13.17 (2012)

### Results and Discussion

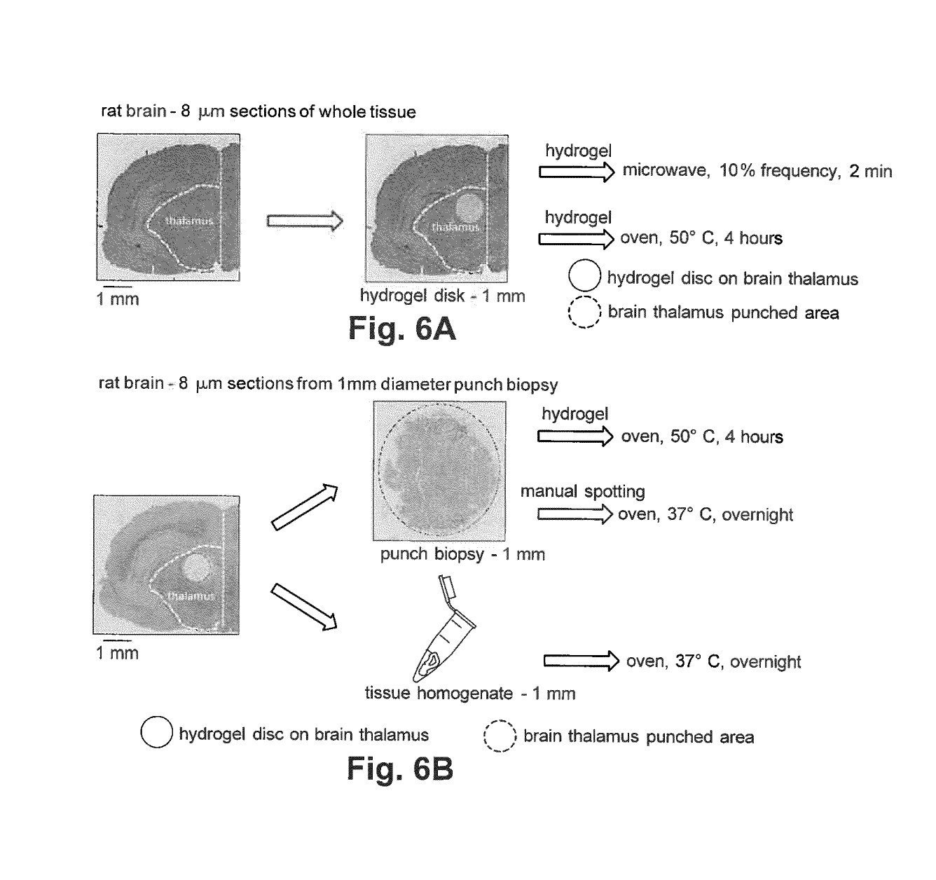

The aims of this study were to demonstrate both the reliability and the relative advantages of the use of microwave radiation to speed up the on-tissue proteomics workflow and to demonstrate the methodology to perform the enzymatic digestion in a histologically defined region on a thin tissue specimen (8 μm). Various on-tissue digestion strategies were carried out on the same sample in this study to provide a basis for comparison; therefore, a series of experiments were designed and carried out using rat brain serial sections from the same tissue specimen, to avoid tissue proteome variability. FIG. 6 shows a graphical depiction of the experimental design used in this study. Fresh frozen rat brain biopsies were first sectioned at 8 μm thickness and then stained by hematoxylin and eosin (H&E) for histological evaluation of regions of interest; the thalamic region was chosen for the histology-directed experiments. Hydrogel discs, prepared on a prior day and stored, were reconstituted in the trypsin solution for 15 minutes and then placed on the rat brain thalamic region as described above. First, two on-tissue digestion experiments were carried out: FIG. 6a shows two different digestion strategies (microwave and conventional heating) performed on the whole tissue specimen using the hydrogel disc for the histology-directed digestion. FIG. 6b displays another set of experiments performed on the rat brain thalamic region punched from the bulk biopsy specimen prior cryosectioning. The bulk specimen was cryosectioned and stained to provide a visual comparison of the areas sampled for analysis. The biopsied tissue from the bulk specimen was cryosectioned at a thickness of 8 μm to collect a precisely comparable amount of tissue for comparison with the on-tissue digestion.

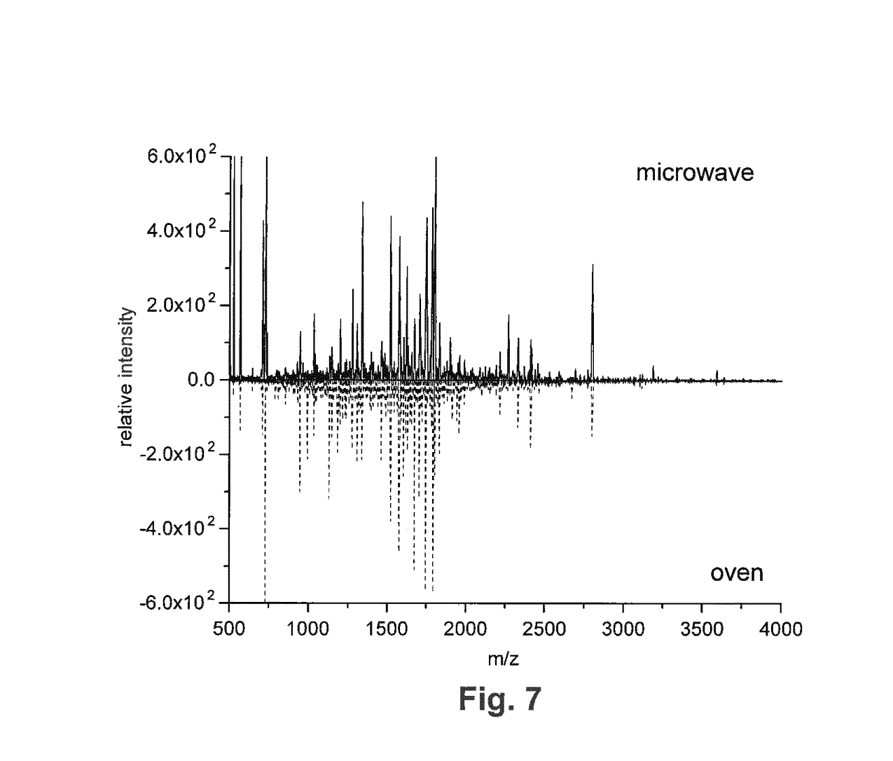

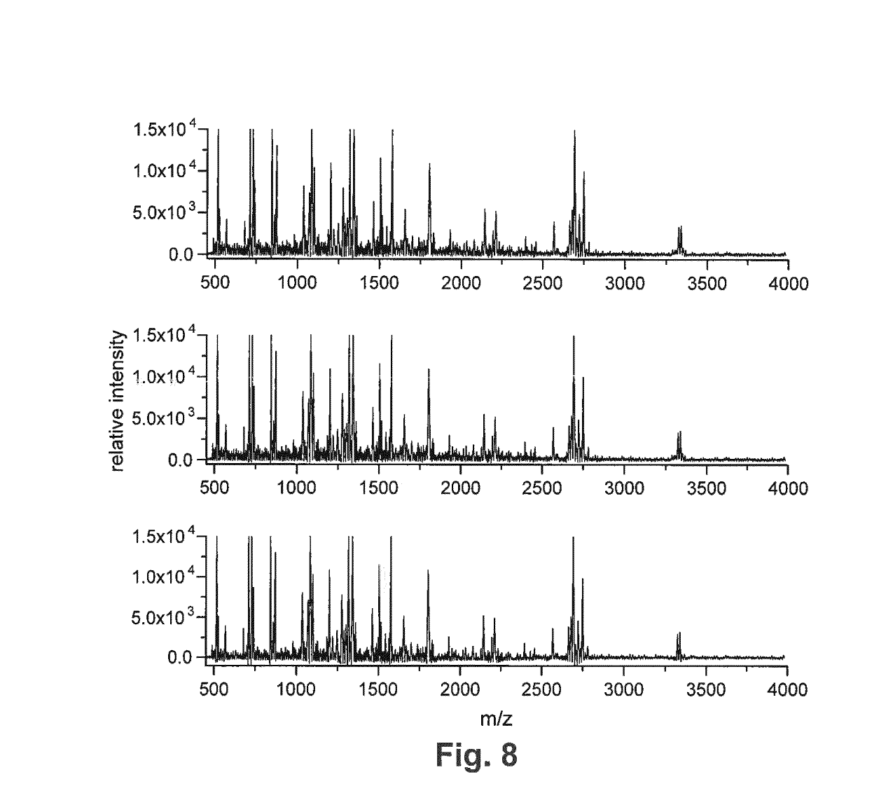

Solvent extracted digested peptides obtained from the first two hydrogel experiments, carried out on-tissue via microwave and oven, were reconstituted and mixed with a solution of CHCA for MALDI MS analyses. The resulting profile MALDI spectra are displayed in FIG. 7. The peptide spectra are qualitative comparable with most major ions present in both preparations in the mass range measured (500-4000 Da). While most peaks are present, there are notable relative intensity differences, likely due to the different incubation strategy (microwave vs. conventional oven). This result should have little impact on downstream identification by LC-MS/MS; however, these differences would not permit quantitative comparisons between samples that have been prepared using two different on-tissue digestion approaches. MALDI MS profiles of three technically replicated microwave assisted hydrogel mediated on-tissue digested extracts are presented in FIG. 8. Most of the signals are detected in all the three replicates, confirming the reliability of the microwave procedure for the digestion.

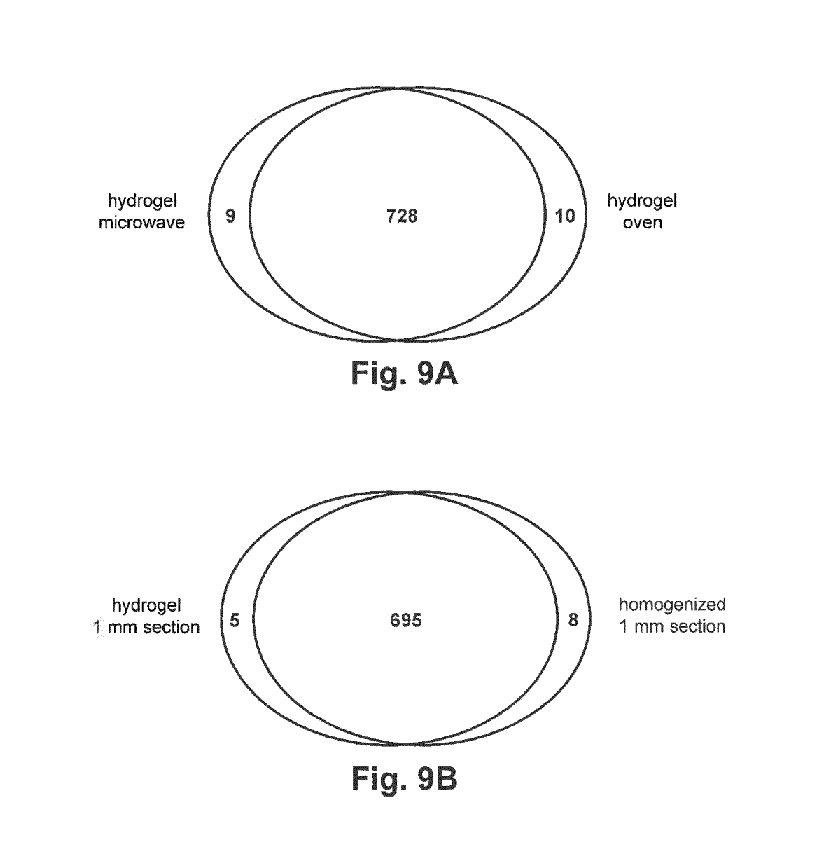

To further validate the on-tissue hydrogel/microwave digestion approach, other on-tissue digestion experiments were carried out as described in FIG. 6b. Serial sections at 8 μm thickness and 1 mm diameter were cut from the thalamic region of rat brain as described in the experimental section. This experiment was performed to further validate the extraction localized proteins by the hydrogel discs through the exposure of a tissue surface cut at the same diameter which the hydrogel discs were fabricated (1 mm). Rat brain thalamus proteins were digested following three approaches: 1) using a hydrogel disc and incubating the reaction for 4 hours at 50° C.; 2) by deposition of the trypsin solution onto the tissue surface and 3) by the homogenization of tissue specimens followed by protein extraction; both the last two approaches were allowed for digestion by overnight incubation at 37° C. (FIG. 6b). All the digested extracts, were analyzed by LC-MS/MS followed by database search for protein identification. Data were processed using a 5% FDR, filtering the identified proteins with number of unique peptides ≧2 and a p-value <0.05. Data are summarized in FIG. 9 using Venn diagrams.

The digestion strategies were evaluated and compared in different ways: the first comparison was carried out considering the number of identified proteins found when using microwave heating compared to conventional heating in an oven. A large number of proteins (728) were identified in both the microwave digestion strategy and the oven incubation, both using the hydrogel disc (FIG. 9a); this finding suggests that the hydrogel disc device allows for a comparable degree of digestion in 2 minutes using microwave heating as well as in 4 hours using conventional heating. Relatively few proteins were uniquely identified in both approaches, respectively nine from the microwave digestion and ten from the oven digestion. This finding confirms that by changing the method of heating to microwave irradiation, the protein population sampled from the brain tissue was not altered. Moreover, FIG. 9b displays a Venn diagram comparing the number of identified proteins within the experiments performed using the 1 mm diameter tissue specimens from the thalamic region of the rat brain. Also in this case, the majority of the identified proteins (695) were identified using both digested extracts (from the hydrogel disc and from the tissue homogenization), while a few proteins were found uniquely expressed into the two set of samples, respectively five from the sample from the hydrogel digestion and eight from the homogenization process (FIG. 9b). Since homogenization is considered to be the most comprehensive method of protein extraction from tissue, allowing for the most complete disruption of tissue and cell architecture among the methods tested, the hydrogel disc method displays a remarkable similarity to the results obtained using the conventional homogenization approach. The manual spotted digestion extracts were evaluated: the number of identified proteins was found similar to the number of identified proteins from the other strategies considered in this study.

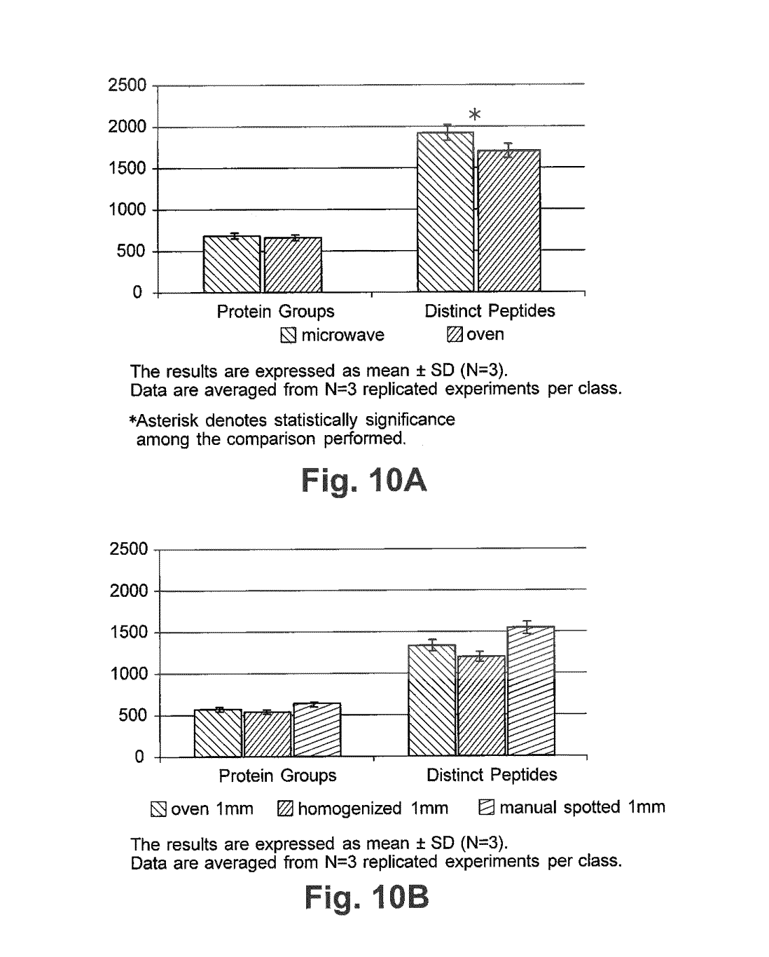

All the different on-tissue digestion strategies were further validated using the LC-MS/MS data from the replicated experiments. Thus, two parameters were considered for the comparison. The first parameter was the number of protein groups, which defines the minimum number of uniquely identified proteins (when several possible proteins have highly similar sequences and cannot be distinguished by the peptides identified in a given experiment, these proteins are reported as a single group). The second parameter used for the evaluation of all the different digestion strategies was the number of distinct peptides: peptides are considered distinct when they identify unique sequences of amino acids. FIG. 10a displays the number of protein groups and of distinct peptides which were obtained from the hydrogel digestion experiments performed using both the microwave for 2 min and the conventional oven for 4 hours: results were found to be very similar for all the metrics compared. These results, along with those of FIG. 9a confirm that accelerating the on-tissue digestion using microwave radiation is reliable and it allows for an almost identical population of proteins to be identified. Moreover, very few differences were found when comparing the number of distinct peptides; however, a statistically significant increase of the peptides identified in the microwave digestion experiments was observed. FIG. 10b illustrates the comparison between the experiments performed on the 1 mm diameter rat brain thalamic region. In this case, the number of protein groups was not significantly changed among all the digestion strategies (FIG. 10b). Besides, considering the number of distinct peptides, the manual spotting digestion strategy gave higher values. Thus, probably the digestion efficiency of these approaches differed. However, given that the same amount of trypsin was used in all the methods, using the hydrogel disc as well as in the classic on-tissue digestions, the difference may be due to the limited volume of the hydrogel and also to the different incubation step (microwave vs. oven). Furthermore, the Mann-Whitney U test (for the hydrogel digestion experiments performed via microwave and oven) and Kruskal-Wallis test (for all the digestions carried out using the 1 mm rat brain tissue specimens) were applied to the number of protein groups and the number of distinct peptides in order to find possible statistical differences between groups. Significant difference was found only for the number of distinct peptides within the comparison microwave/oven (p<0.05).

### CONCLUSIONS

The inventors have developed a method to significantly speed up on-tissue protein digestion by applying microwave irradiation for two minutes. This method can be used for histology-directed analysis since it utilizes hydrogel discs fabricated at 1 mm diameter that are precisely placed on defined regions, localizing the digestion to a defined area of the tissue. The reliability and reproducibility of the microwave assisted digestion has been demonstrated by the comparison of the number of identified proteins and other data from the LC-MS/MS experiments. This study demonstrates that a rapid and reliable protein identification strategy can be performed on a single tissue specimen while preserving the inherent spatial information of the tissue. This is of primary importance when the amount of material (tissue biopsy) is often not enough for proteomics as well as for all the other analysis that are usually carried out on a biopsy for clinical investigations. In contrast, the conventional tissue homogenization and digestion procedures are slower, more time consuming, and have a significantly higher number of steps. This often results in the need for a higher amount of starting material because of sample loss resulting from handling of the tissue. Moving forward, the hydrogel discs fabrication can be optimized for different dimensions, according to tissue regions of interest. Future work will also include optimization of the hydrogel methodology for multiple enzymes experiment as well as for intact protein analyses. Taken together, these results suggest the possible clinical utility of histology-directed protein digestion approach.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method for analyzing the polypeptide content of animal tissue, comprising:
(a) providing an animal tissue specimen;
(b) depositing one or more portions of a hydrogel mixture including a protease on spatially discrete portions of the animal tissue specimen;
(c) allowing sufficient time to pass for animal tissue under the hydrogel mixture to form a digested mixture of animal tissue and hydrogel mixture;
(d) removing the digested mixture from the animal tissue and extracting polypeptides from the digested mixture to provide an extract; and
(e) analyzing the polypeptide content of the extract by mass spectrometry.

2. The method of claim 1, wherein a plurality of portions of hydrogel mixture are deposited to provide a plurality of extracts that are analyzed by mass spectrometry.

3. The method of claim 2, wherein the polypeptide content from a plurality of spatially discrete regions of the animal tissue specimen are compared.

4. The method of claim 1, wherein the animal tissue is diseased or injured.

5. The method of claim 1, wherein the animal tissue is heart tissue, liver tissue, kidney tissue, prostate tissue, breast tissue, ovary tissue, uterine tissue, skin tissue, lung tissue, brain tissue, colon tissue, pancreatic tissue, or muscle tissue.

6. The method of claim 1, wherein the hydrogel is an ionotropic hydrogel.

7. The method of claim 1, wherein the hydrogel comprises an alginate or polyacrylamide polymer.

8. The method of claim 1, wherein the protease is trypsin.

9. The method of claim 1, wherein the animal tissue specimen has a thickness ranging from about 5 μm to about 50 μm.

10. The method of claim 1, wherein the hydrogel portions are drops or discs.

11. The method of claim 10, wherein the drops or discs have a diameter ranging from about 250 μm to about 1 mm.

12. The method of claim 1, wherein the hydrogel portions are prepared using a paper template.

13. The method of claim 1, wherein the mass spectrometry is MALDI mass spectrometry or LC-MS/MS.

14. The method of claim 1, wherein the hydrogel mixture is heated during formation of the digested mixture.

15. The method of claim 13, wherein the hydrogel mixture is heated using microwave radiation.

16. The method of claim 1, wherein one or more steps of the method are automated.
